(19) United States
(12) Patent Application Publication
(10) Pub. No.: US 2023/0049319 A1
(43) Pub. Date: Feb. 16, 2023

(54) CANNABINOID-COMPRISING COSMETIC COMPOSITIONS

(71) Applicant: THIS WORKS PRODUCTS LIMITED, Wimbledon (GB)

(72) Inventor: Anna PERSAUD, Wimbledon (GB)

(21) Appl. No.: 17/642,088

(22) PCT Filed: Mar. 8, 2021
(86) PCT No.: PCT/IB2021/051919

§ 371 Date: Mar. 10, 2022

Related U.S. Application Data
(60) Provisional application No. 63/019,855, filed on May 4, 2020.

(51) Int. Cl. A61K 8/49; A61K 8/67; A61K 8/34; A61Q 19/08; A61K 8/35; A61Q 19/00; A61Q 19/10

(52) U.S. Cl. CPC A61K 8/498; A61K 8/678; A61K 8/347; A61Q 19/08; A61K 8/355; A61K 8/676; A61Q 19/007; A61Q 19/10

(57) ABSTRACT

The present technology generally relates to compositions comprising cannabidiol (CBD) or a derivative thereof and at least one active cosmetic ingredient, for use in cosmetics and personal care products such as anti-aging or moisturizing creams for skin care and massage oils for muscle therapy.

### CROSS-REFERENCE TO RELATED APPLICATION

This application claims the benefit of and priority to U.S. provisional patent application No. 63/019,855, filed on May 4, 2020; the content of which is herein incorporated in entirety by reference.

### FIELD

The present technology generally relates to cannabinoid compositions comprising at least one purified cannabidiol (CBD) and at least one active cosmetic ingredient, for use in cosmetic and personal care products, and methods of use thereof.

### BACKGROUND

Cannabis comprises hundreds of compounds, including phytocannabinoids that can modulate various components of the central nervous system (CNS) including the ECS, and serotonergic activity. In Cannabis, over 100 phytocannabinoids have been identified, although only two: 9-Δ-tetrahydrocannabinol (THC) and cannabidiol (CBD) have been thoroughly investigated. THC is considered psychoactive. In contrast to THC, CBD is a non-psychoactive cannabinoid and is found in both cannabis and industrial hemp. CBD is known to have anxiolytic, anti-inflammatory, antioxidant, and antimicrobial properties.

Cannabinoids mediate their effects through cannabinoid receptors. Natural or synthetic cannabinoid receptor agonists have been shown to be of therapeutic value for a number of important medical conditions, including anxiety, pain, insomnia, and depression, as well as substance abuse. Cannabinoid receptors include CB₁, which is predominantly expressed in the brain, and CB₂, which is primarily found on the cells of the immune system and expressed in several types of inflammatory cells and immunocompetent cells. CBD was shown to substantially reduce chronic inflammation and pain in mice and rats (Xiong, W. et al., J. Exp. Med. 209(6): 1121-1134, 2012).

Researchers have suggested that CBD could help reduce acne by curbing the production of sebum that leads to unwanted breakouts (Olah, A. et al., J. Clin. Invest. 124(9): 3713-3724, 2014). It is also known that CBD can be absorbed transcutaneously, consistent with the observation that human skin has native receptors for cannabinoids. Cannabinoids such as CBD therefore show great promise for treating skin conditions such as acne and eczema and other related conditions.

It has been suggested therefore that cannabis-infused beauty products containing Cannabidiol (CBD) could provide beneficial therapeutic and cosmetic effects, however products with high enough levels of active ingredients to yield measurable results have not been successfully formulated. There remains a need for cannabis-infused cosmetic and personal care products that can combine high levels of potent cosmetic ingredients such as retinoids, alpha hydroxy acids, vitamins, essential oils, and the like, and cannabinoids, for improving skin appearance and treating skin conditions.

### SUMMARY

The present disclosure provides compositions and methods of using the compositions for, inter alia, improving skin appearance and treating inflammatory skin conditions such as acne. The compositions comprise compounds found in cannabis and active cosmetic ingredients. Compositions may also comprise additional non-medicinal cosmetic ingredients such as antioxidants to stabilize the compositions, for example by preventing discoloration. In some cases, compositions may also comprise functional fragrances for aromatherapy.

In a broad aspect, there is provided a composition for improving the appearance of skin, the composition comprising at least one cannabinoid or a derivative thereof, and at least one active cosmetic ingredient. In some embodiments, the at least one cannabinoid is cannabidiol (CBD). The at least one cannabinoid may be present in the composition in an amount ranging from about 0.5 wt % to about 10 wt %, or from about 0.5 wt % to about 5 wt %, or from about 1 wt % to about 5 wt %, or from about 1 wt % to about 2 wt %, about 2 wt % to about 3 wt %, about 3 wt % to about 5 wt %, or in about 1 wt %, about 3 wt %, or about 5 wt % of the composition. The cannabinoid may be purified and may be isolated from Cannabis or synthetically derived.

In some embodiments, the at least one active cosmetic ingredient comprises Ascorbyl Glucoside, Ubiquinone, one or more alpha hydroxy acid (AHA), Bakuchiol, Hydroxypinacolone Retinoate (HPR), or a combination thereof. The one or more AHA may be, for example and without limitation, glycolic acid, citric acid, tartaric acid, lactic acid, or a combination thereof. In some embodiments, the composition further comprises one or more of: Caprylic/Capric Triglyceride, Squalane, Sodium hyaluronate, one or more amino acid, sodium lactate, glycerin, Aloe vera, Aloe barbadensis Leaf Juice Powder, one or more essential oil, Glyceryl stearate, Octyldodecanol, PCA, Sodium PCA, ethylhexylglycerin, lecithin, BHA, BHT, derivatives thereof, and combinations thereof. Compositions may also comprise one or more purified terpene such as, without limitation, linalool, linalool oxide, limonene, geraniol, citronellol, eugenol, citral, farnesol, derivatives thereof, or combinations thereof.

In some embodiments, the composition of the present technology is formulated as a bi-phasic formulation in which the oil and water phases are separated, allowing high loading of the active ingredients in a stabilized composition.

In some embodiments, the composition of the present technology comprises at least one antioxidant, such as tocopherol, to stabilize the composition by, e.g., preventing discoloration. The antioxidant may for example be present in the composition in an amount ranging from about 0.05 wt % to about 1 wt %.

In some embodiments, the composition is in the form of an ointment, a cream, a suspension, a liniment, a balm, a serum, a lotion, a paste, a gel, a spray, an aerosol, a foam, an oil, a liquid, a powder, a patch, a matrix, or a wax. Compositions may be for administration topically, transdermally, by inhalation, or nasally.

In another broad aspect, there is provided a cosmetic or a personal care product comprising the composition according to the present technology. The cosmetic or personal care product may be, for example and without limitation, a skin care product or makeup, useful for anti-aging, anti-wrinkle, and/or anti-acne treatments, for moisturizing or hydrating skin, for exfoliation, for reducing redness, pigmentation or discoloration, and/or for improving overall skin appearance such as skin texture, color or tone.

In yet another broad aspect, there is provided a method for improving appearance of a skin area of a subject, the method comprising administering to the subject an effective amount of the composition of the present technology, such that the appearance of the skin area is improved. For example, the composition may improve the skin area's texture, tone or color; reduce appearance of wrinkles or fine lines; and/or reduce erythema or redness.

Another aspect of the present disclosure is directed to a method for moisturizing or hydrating skin of a subject, the method comprising administering to the subject an effective amount of the composition of the present technology, such that the skin of the subject is moisturized or hydrated.

Another aspect of the present disclosure is directed to a method for treating an inflammatory skin condition in a subject in need thereof, the method comprising administering to the subject an effective amount of the composition of the present technology, such that the inflammatory skin condition is treated. Inflammatory skin conditions that may be treated include, for example and without limitation, psoriasis, eczema, acne and cold sores.

Methods for relieving muscle pain in a subject in need thereof are also provided, the methods comprising administering to the subject an effective amount of the composition of the present technology, such that the subject has relief of muscle pain.

It is further contemplated that various kits and dosage devices will be provided for use with the compositions of the disclosure.

Other aspects and features of the present disclosure will become apparent to those ordinarily skilled in the art upon review of the following description of specific embodiments.

### DETAILED DESCRIPTION

The present technology is explained in greater detail below. This description is not intended to be a detailed catalog of all the different ways in which the technology may be implemented, or all the features that may be added to the instant technology. For example, features illustrated with respect to one embodiment may be incorporated into other embodiments, and features illustrated with respect to a particular embodiment may be deleted from that embodiment. In addition, numerous variations and additions to the various embodiments suggested herein will be apparent to those skilled in the art in light of the instant disclosure which variations and additions do not depart from the present technology. Hence, the following description is intended to illustrate some particular embodiments of the technology, and not to exhaustively specify all permutations, combinations and variations thereof.

As used herein, the singular form “a,” “an” and “the” include plural referents unless the context clearly dictates otherwise.

The recitation herein of numerical ranges by endpoints is intended to include all numbers subsumed within that range (e.g., a recitation of 1 to 5 includes 1, 1.25, 1.5, 1.75, 2, 2.45, 2.75, 3, 3.80, 4, 4.32, and 5).

The term “about” is used herein explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value. For example, the term “about” in the context of a given value or range refers to a value or range that is within 20%, preferably within 15%, more preferably within 10%, more preferably within 9%, more preferably within 8%, more preferably within 7%, more preferably within 6%, and more preferably within 5% of the given value or range.

The expression “and/or” where used herein is to be taken as specific disclosure of each of the two specified features or components with or without the other. For example, “A and/or B” is to be taken as specific disclosure of each of (i) A, (ii) B and (iii) A and B, just as if each is set out individually herein. The term “or” as used herein should in general be construed non-exclusively. For example, an embodiment of “a composition comprising A or B” would typically present an aspect with a composition comprising both A and B. “Or” should, however, be construed to exclude those aspects presented that cannot be combined without contradiction (e.g., a composition pH that is between 9 and 10 or between 7 and 8).

As used herein, the term “comprise” is used in its non-limiting sense to mean that items following the word are included, but items not specifically mentioned are not excluded.

As used herein, the term “purified” means extracted, isolated, and/or separated from other compounds, formulations, compositions, matter, and/or mass resulting in a greater than 60% purity.

In some embodiments a “purified” cannabinoid (or “purified” terpene) is greater than about 70% pure, greater than about 75% pure, greater than about 80% pure, greater than about 85% pure, greater than about 90% pure, greater than about 91% pure, greater than about 92% pure, greater than about 93% pure, greater than about 94% pure, greater than about 95% pure, greater than about 96% pure, greater than about 97% pure, greater than about 98% pure, or greater than about 99% pure. Within the context of the present disclosure, where a compound comprises stereogenic centers, the term “purified” includes enantiomerically pure compositions and also mixtures of enantiomers or isomers.

Also within the context of the present disclosure, purified compounds may be purposely formulated with other compounds at various levels of purity. Provided that the ingredients used for purposeful formulation are purified prior to the said purposeful formulation, the act of subsequently formulating them does render them not “purified” within the context of an ingredient list.

In an embodiment, the term “purified” may refer to a cannabinoid that is separated from plant matter from which it was derived.

In an embodiment, the term “purified” may refer to a terpene that is separated from plant matter from which it was derived.

As used herein, the term “Cannabis” refers to the genus of flowering plants in the family Cannabaceae. Three species may be recognized as being part of the Cannabis genus, namely: Cannabis sativa, Cannabis indica, and Cannabis ruderalis. The expressions “Cannabis sativa” and “C. sativa” are used herein interchangeably. The term “strain” as used herein refers to different varieties of the plant genus Cannabis. For example, the term “strain” can refer to different pure or hybrid varieties of cannabis plants. In some instances, the cannabis strain of the present technology can by a hybrid of two strains, for example, a hybrid between C. sativa and C. indica. Different cannabis strains often exhibit distinct chemical compositions with characteristic levels of cannabinoids and terpenes, as well as other components. Differing cannabinoid and terpene profiles associated with different cannabis strains can be useful for the treatment of different diseases, or for treating different subjects with the same disease.

The expression “cannabis oil” as used herein refers to a mixture of compounds obtained from the extraction of cannabis plants. Such compounds include, but are not limited to, cannabinoids, terpenes, terpenoids, and other compounds found in the cannabis plant. The exact composition of cannabis oil depends on the strain of cannabis that is used for extraction, the efficiency and process of the extraction itself, and any additives that might be incorporated to alter the palatability or improve administration and/or bioavailability of the cannabis oil.

The term “eluate” as used herein refers to a solution that is collected after contacting a plant material, such as raw cannabis plant material, with an extraction solvent. The eluate can contain dissolved cannabinoids as well as other compounds. The term “filtrate” refers to a solution that has passed through a membrane or strainer of variable porousness or permeability to remove either particulate matter or unwanted compounds. As used herein, the term “distillate” refers to a solution that has been concentrated by any known means of evaporation or distillation. In some embodiments of the present technology, the filtrate is evaporated to form the distillate. The term “extract” as used herein refers to a solution that has been purged or dehydrated to remove residual solvent. In some embodiments of the present technology, the extract is formed by purging or dehydrating the distillate using any known means in the art. As used herein, the term “isolate” refers to a chemical substance that has been separated from foreign or contaminating substances. Pure results of a successful purification process are termed isolate. In some embodiments of the present technology, the isolate is refined distillate.

As used herein, the term “cannabinoid” refers to a chemical compound belonging to a class of secondary compounds commonly found in plants of genus cannabis, but also encompasses synthetic and semi-synthetic cannabinoids.

The most notable cannabinoid is tetrahydrocannabinol (THC), the primary psychoactive compound in cannabis. Cannabidiol (CBD) is another cannabinoid that is a major constituent of the phytocannabinoids. There are at least 113 different cannabinoids isolated from cannabis, exhibiting varied effects.

Synthetic cannabinoids and semi-synthetic cannabinoids encompass a variety of distinct chemical classes, for example and without limitation: the classical cannabinoids structurally related to THC, the non-classical cannabinoids (cannabimimetics) including the aminoalkylindoles, 1,5 diarylpyrazoles, quinolines, and arylsulfonamides as well as eicosanoids related to endocannabinoids.

In many cases, a cannabinoid can be identified because its chemical name will include the text string “*cannabi*”. However, there are a number of cannabinoids that do not use this nomenclature.

Within the context of this disclosure, where reference is made to a particular cannabinoid, each of the acid and/or decarboxylated forms are contemplated as both single molecules and mixtures. In addition, salts of cannabinoids are also encompassed, such as salts of cannabinoid carboxylic acids.

As well, any and all isomeric, enantiomeric, or optically active derivatives are also encompassed. In particular, where appropriate, reference to a particular cannabinoid includes both the “A Form” and the “B Form”. For example, it is known that THCA has two isomers, THCA-A in which the carboxylic acid group is in the 1 position between the hydroxyl group and the carbon chain (A Form) and THCA-B in which the carboxylic acid group is in the 3 position following the carbon chain (B Form).

As used herein, the expression “cannabinoid concentrate” refers to products made from the cannabis plant that have been processed to keep only the most desirable plant compounds (primarily the cannabinoids), while removing excess plant material and other impurities. As used herein, the expression “cannabinoid concentrate” includes one or more of cannabinoid distillate and cannabinoid isolate (e.g., crystalline CBD).

As used herein, the term “endocannabinoid” refers to the biological system composed of endocannabinoids, which are endogenous lipid-based retrograde neurotransmitters that bind to cannabinoid receptors, and cannabinoid receptor proteins that are expressed throughout the vertebrate central nervous system (including the brain) and peripheral nervous system.

The expression “cannabinoid receptor” refers hereinafter to a class of cell membrane receptors under the G protein-coupled receptor superfamily There are currently two known subtypes of cannabinoid receptors, termed CB₁ and CB₂. The CB₁ receptor is expressed in the brain, the lungs, the liver, the kidneys, and throughout the body. The CB₂ receptor is expressed mainly in the immune system and in hematopoietic cells. The expression “Cannabinoid receptor type 1 (CB₁)” refers hereinafter to a G protein-coupled cannabinoid receptor located primarily in the central and peripheral nervous system. It is activated by the endocannabinoid neurotransmitters anandamide and 2-arachidonoyl glyceride (2-AG); by plant cannabinoids, such as the compound THC, an active ingredient of the psychoactive drug cannabis; and by synthetic analogues of THC. The expression “Cannabinoid receptor type 2 (CB₂)” refers hereinafter to a G protein-coupled receptor from the cannabinoid receptor family that in humans is encoded by the CNR2 gene. It is closely related to the cannabinoid receptor type 1, which is largely responsible for the efficacy of endocannabinoid-mediated presynaptic-inhibition, the psychoactive properties of Tetrahydrocannabinol, the active agent in marijuana, and other phytocannabinoids (natural cannabinoids).

As used herein, the expression “effective amount” or “therapeutically effective amount” refers to the amount of components of the compositions of the present disclosure which are effective for producing some desired therapeutic effect as defined herein at a reasonable benefit/risk ratio applicable to any treatment.

As used herein, the term “anxiolytic” or “anti-anxiety” refers to an agent or other intervention that inhibits anxiety. This effect is in contrast to anxiogenic agents, which increase anxiety. Together these categories of psychoactive compounds or interventions may be referred to as anxiotropic compounds or agents. Some recreational drugs such as alcohol (also known as ethanol) induce anxiolysis initially; however, studies show that many of these drugs are anxiogenic.

In one embodiment, the present technology relates to compositions comprising at least one cannabinoid compound, e.g., CBD or a derivative thereof, and at least one active cosmetic ingredient, e.g, Ascorbyl Glucoside, Ubiquinone, one or more alpha hydroxy acid (AHA), Bakuchiol, Hydroxypinacolone Retinoate (HPR), or a combination thereof. In some implementations of this embodiment, the compositions of the present disclosure are useful in methods of, inter alia, improving appearance of a skin area of a subject, moisturizing or hydrating skin of a subject, and treating inflammatory skin conditions in a subject.

i) Cannabinoids

In some embodiments, the cannabinoid present in the compositions of the present technology is selected from one or more of: Cannabigerol-type (CBG): cannabigerol ((E)-CBG C-5), cannabigerol monomethyl ether ((E)-CBGM C-5A), Cannabinerolsäure A ((Z)-CBGA C-5A), Cannabigerovarin (((e)-BGV C-3), Cannabigerolsäure A(e)-CBGA C-5A), A Cannabigerolsäure monomethyl ether ((e)-CBGAM C-5A), Cannabigerovarinsäure A ((e)-CBGVA-C3A); Cannabichromene-type (CBC): cannabichromene (CBC-C5), Cannabichromensäure A (CBCA C-5A), Cannabichromevarin (CBCVC-3), Cannabichromevarinsäure A (CBCVA-C3A); Cannabidiol-type (CBD), cannabidiol (CBD-C5), cannabidiol monomethyl (CBDM-C5), cannabidiol-C4 (CBD-C4), Cannabidivarin (CBDV-C3), Cannabidiorcol (CBD-C1), cannabidiolic (CBDA C-5), Cannabidivarinsäure (CBDVA C-3); Cannabinodiol-like (CBND): Cannabinodiol (CBND C-5), Cannabinodivarin (CBND C-3); Tetrahydrocannabinol-like (THC): Δ⁹-tetrahydrocannabinol (Δ⁹-THC-C5), Δ⁹-tetrahydrocannabinol-C4 (Δ⁹-THC-C4), Δ⁹-tetrahydrocannabivarin (Δ⁹-THCV-C3), Δ⁹-Tetrahydrocannabiorcol (Δ⁹-THCO C-1), Δ⁹-Tetrahydrocannabinolsäure (Δ⁹ THCA-C-5A), Δ⁹-Tetrahydrocannabinolsäure B (Δ⁹ THCA-C-5B), Δ⁹-Tetrahydrocannabinolsäure-C4 (Δ⁹ THCA-C-4A and/or B), Δ⁹-Tetrahydrocannabivarinsäure A (Δ⁹-THCVA-C3A), Δ⁹-Tetrahydrocannabiorcolsäure (Δ⁹-THCOA-C1 A and/or B), (−)-Δ⁸-trans-(6aR, 10aR)-8-tetrahydrocannabinol (Δ⁸-THC-C 5), (−)-Δ⁸-trans-(6aR, 10aR)-Tetrahydrocannabinolsäure A (Δ⁸-THCA-C 5A); (−)-(6a S, 10a R)-Δ⁹-tetrahydrocannabinol ((−)-cis-Δ⁹-THC-C5); Cannabinol-type (CBN): Cannabinol CBN-C5, cannabinol C4 (CBN-C4), Cannabivarin (CBN-C3), cannabinol C2 (CBN-C2), Cannabiorcol (CBN-C1), Cannabinolsäure A (C5 CBNA-A), Cannabinolmethylether (CBNM C-5) Cannabitriol-type (CBT): (−)-(9R,10R)-trans-Cannabitriol ((−)-trans-CBT-C5), (+)-(9S,10S)-Cannabitriol ((+)-trans-CBT C-5), (±)-(9R, 10S/9S, 10R)-Cannabitriol ((±)-cis-CBT-C5), (−)-(9R,10R)-trans [10-0-thyl-cannabitriol] ((−)-trans-CBT-OEt-C5), (±)-(9R, 10R/9S, 10S)-Cannabitriol-C3 ((±)-trans-CBT-C3), 8,9-dihydroxy-46a (10a) tetrahydrocannabinol (8,9-di-OH-CBT-C5), cannabidiolic A (CBDA C-59-OH-CBT-C5 ester), (−)-(6aR, 9S, 10S, 10aR)-9,10-dihydroxy-hexahydrocannabinol, Cannabiripsol Cannabiripsol-C5, (−)-6a,7,10a-trihydroxy-Δ⁹-tetrahydrocannabinol ((−)-Cannabitetrol), 10-oxo-46a (10a) tetrahydrocannabinol (OTHC); Cannabielsoin-like (CBE): (5aS, 6S, 9R, 9aR)-C5-Cannabielsoin (CBEC-5), (5aS, 6S, 9R, 9aR)-C3-Cannabielsoin (CBE C-3), (5aS, 6S, 9R, 9aR)-Cannabielsoinsäure A (CBEA-C5 A), (5aS, 6S, 9R, 9aR)-Cannabielsoinsäure B (CBEA-C5 B), (5aS, 6S, 9R, 9aR)-C3 Cannabielsoinsäure B (CBEA-C3 B), Cannabiglendol-C3 (OH-iso-HHCV C-3), Dehydrocannabifuran (DCBF C-5), Cannabifuran (CBF-C5); Isocannabinoide: (−)-Δ⁷-trans-(1R, 3R, 6R)Isotetrahydrocannabinol, (±)-Δ⁷-1,2-cis-(1R, 3R, 6S/1S, 3S, 6R)-Isotetrahydrocannabivarin, (−)-Δ7-trans-(1R, 3R, 6R)-Isotetrahydrocannabivarin; Cannabicyclol-like (CBL): (±)-(1aS, 3aR, 8bR, 8Cr-cannabicyclol (CBL-C), (±)-(1aS, 3aR, 8bR, 8Cr-Cannabicyclolsäure A (CBLA-C5A) (±)-(1aS, 3aR, 8bR, 8Cr-Cannabicyclovarin (CBLV C-3); Cannabicitran-type (CBT): Cannabicitran (CBT-C5); Cannabichromanon-like (CBCN): Cannabichromanon (CBCN C-5), Cannabichromanon-C3 (CBCN C-3), Cannabicoumaronon (CBCON C-5). In addition to the above cannabinoids, the carboxylic acids which are biosynthetic precursors of each are contemplated as cannabinoids which may be present in the compositions of the present technology.

In some implementations of these embodiments, the compositions of the present technology comprise one or more of: CBD, THC, CBG, CBN, CBC, THCV, CBGA, CGCA, CBCA, THCA and CBDA.

In some implementations of these embodiments, the compositions of the present technology may comprise a predominant cannabinoid and minimal or trace amounts of other cannabinoids. For example, the composition of the present technology may comprise CBD as a predominant cannabinoid and trace amounts or minimal amounts of THC.

As used herein, the expression “CBD:THC ratio” or “THC:CBD ratio” refers to the amount of CBD and THC that is comprised in any of the compositions of the present technology. THC dominant options (0:1) will provide varying degrees of psychoactivity. CBD dominant ratios of 100:1, 25:1 or 1:0 offer little or no psychoactivity.

In some embodiments, the compositions of the present technology comprise cannabinoid in an amount ranging from between about 0.001 wt % to about 99.999 wt %, between about 0.001 wt % and about 90 wt %, between about 0.01 wt % and about 90 wt %, between about 0.1% wt % and about 90 wt %, between about 0.001 wt % to about 75 wt %, between about 0.001% and about 75 wt %, between about 0.01 wt % and about 75 wt %, between about 0.1% wt % and about 75 wt %, between about 0.001 wt % to about 50 wt %, between about 0.001 wt % and about 50 wt %, between about 0.01 wt % and about 50 wt %, between about 0.1% wt % and about 50 wt %, between about 0.001 wt % to about 25 wt %, between about 0.001 wt % and about 25 wt %, between about 0.01 wt % and about 25 wt %, between about 0.1% wt % and about 25 wt %, between about 0.001 wt % and about 20 wt %, between about 0.5 wt % and about 10 wt %, between about 1 wt % and about 5 wt %, between about 2 wt % and about 5 wt %, between about 3 wt % and about 5 wt %, between about 1 wt % and about 3 wt %, between about 2 wt % and about 3 wt %, about 1 wt %, about 2 wt %, about 3 wt %, about 4 wt %, about 5 wt %, about 6 wt %, about 7 wt %, about 8 wt %, about 9 wt % or about 10 wt % of the total weight of the composition.

In some implementations, the cannabinoid is cannabidiol (CBD) or a derivative thereof selected from CBD, CBDV, CBDA and any combination thereof and wherein the CBD or a derivative thereof is selected from natural CBD or a derivative thereof produced in the body of humans and animals, CBD or a derivative thereof extracted from plants, synthetic CBD or a derivative thereof, and any combination thereof.

In some embodiments, the compositions of the present technology comprise CBD in an amount of from about 0.5 wt % to about 10 wt %, from about 0.5 wt % to about 5 wt %, from about 1 wt % to about 5 wt %, from about 1 wt % to about 2 wt %, from about 2 wt % to about 3 wt %, from about 3 wt % to about 5 wt %, about 0.5 wt %, about 1 wt %, about 2 wt %, about 3 wt %, about 4 wt %, about 5 wt %, about 6 wt %, about 7 wt %, about 8 wt %, about 9 wt % or about 10 wt % of the total weight of the composition.

In various embodiments provided herein, percent refers to percent by weight. In one embodiment, the cannabis isolate or extract comprises about 95% or more of at least one desired cannabinoid. In one embodiment, the cannabis isolate or extract comprises about 90% or more of at least one desired cannabinoid. In one embodiment, the cannabis isolate or extract comprises about 80% or more of at least one desired cannabinoid. In one embodiment, the cannabis isolate or extract comprises about 70% or more of at least one desired cannabinoid. In one embodiment, the cannabis isolate or extract comprises about 60% or more of at least one desired cannabinoid. In one embodiment, the cannabis isolate or extract comprises about 50% or more of at least one desired cannabinoid. In one embodiment, the cannabis isolate or extract comprises about 40% or more of at least one desired cannabinoid. In one embodiment, the cannabis isolate or extract comprises about 30% or more of at least one desired cannabinoid. In one embodiment, the cannabis isolate or extract comprises about 20% or more of at least one desired cannabinoid. In one embodiment, the cannabis isolate or extract comprises about 10% or more of at least one desired cannabinoid. In one embodiment, the cannabis isolate or extract comprises about 5% or more of at least one desired cannabinoid. In one embodiment, the cannabis isolate or extract comprises about 1% or more of at least one desired cannabinoid. In one embodiment, the cannabis isolate or extract comprises about 0.1% or more of at least one desired cannabinoid. In one embodiment, the cannabis isolate or extract comprises about 0.01% or more of at least one desired cannabinoid. In one embodiment, the cannabis isolate or extract comprises about 0.001% or more of at least one desired cannabinoid. In one aspect, the cannabis isolate or extract is diluted to a desired cannabinoid concentration.

In some embodiments, the composition as defined herein is non-psychoactive and does not exhibit any psycho-effect on the subject.

Cannabinoids may be extracted from Cannabis plants or parts thereof using various solvents and technologies including, but not limited to, ethanol, butane, methane, heptane, carbon dioxide, ice, water, and steam. A cannabinoid of interest may be extracted from Cannabis plants bred to express a desired profile of the cannabinoid of interest for purity. In some implementations, the cannabinoid may be extracted using supercritical fluid (SFC) extraction and similar technologies. In some instances, the cannabinoid is crystalized. The process of crystallization involves placing the compound of interest in a liquid and then cooling or adding participants to the solution which would lower the solubility of the compound of interest so that it forms crystals. In this example, crystals are then separated from the liquid through filtration or centrifugation.

In some embodiments, the cannabinoid useful in the compositions of the present technology is a synthetic cannabinoid or is a biosynthetic cannabinoid.

In some embodiments, the cannabinoid useful in the compositions of the present technology is a purified cannabinoid. As used herein, the term “purified” means greater than about 60% pure, greater than about 70% pure, greater than about 80% pure, greater than about 90% pure, greater than about 91% pure, greater than about 92% pure, greater than about 93% pure, greater than about 94% pure, greater than about 95% pure, greater than about 96% pure, greater than about 97% pure, greater than about 98% pure, or greater than about 99% pure.

In some implementations of these embodiments, the cannabinoid is cannabidiol (CBD) or a derivative thereof. Derivatives of cannabidiol that may be used in the compositions of the present technology include compounds that can be imagined to arise or actually be synthesized from a cannabidiol by replacement of one atom of its structure with another atom or with a group of atoms. An example of a derivative of CBD is cannabidiol dimethyl heptyl (CBD-DMH), including all isomeric forms thereof.

ii) Active Cosmetic Ingredients

As used herein, the term “active cosmetic ingredient” refers to a compound that enhances health or well-being of a subject, or makes a subject look, smell or feel better. Examples of active cosmetic ingredients include, without limitation, antioxidants, anti-aging agents, anti-wrinkle agents, anti-inflammatory agents, anti-acne agents, anti-anxiety agents, agents that provide improved sleep, relaxation and/or calm, exfoliants, emollients, moisturizers, and skin-conditioning agents. An active cosmetic ingredient may be, without limitation, a retinoid, a vitamin, an alpha-hydroxy acid, a functional fragrance, an essential oil, an aromatherapeutic, a botanical ingredient, and the like.

Active cosmetic ingredients can have a wide range of uses and advantageous properties. For example, they may be used for their anti-aging, anti-wrinkle, anti-acne, antioxidant, and/or anti-inflammatory properties. In some cases, they may be used for moisturizing (hydrating the skin), for example by increasing the water content of the skin, providing a barrier against moisture loss from the skin, and/or replacing oils contained in the skin. They may also be used to soothe the skin, reduce redness or irritation, give the skin a smooth or fresh appearance, and the like. In addition to the active cosmetic ingredients discussed here, other ingredients may be used to improve the stability, shelf-life, texture, smell, look, skin penetration, etc. of a cosmetic or personal care product; such ingredients are often referred to as non-medicinal ingredients and are discussed further below.

In some embodiments, an active cosmetic ingredient is a botanical ingredient, i.e., an ingredient that originates from plants such as herbs, roots, flowers, fruits, leaves, or seeds. Plant-derived botanical ingredients were among the very first cosmetics as natural colorants, juices for soothing and protection from insect pests, and fragrant oils for imparting odor were all used centuries ago. The actual composition of a botanical ingredient can depend on a number of variables such as the method by which it is prepared. For example, a botanical ingredient may be obtained from a plant part by extraction, such as with the use of a suitable solvent, or directly, such as by drying the plant part or grinding it into a powder, or by squeezing or pressing the plant part to obtain a juice or oil. Most applications of botanical ingredients in cosmetics are topical; they are typically applied externally to the skin or hair.

In some embodiments, therefore, an active cosmetic ingredient is obtained from natural sources, e.g., plant-derived or animal-derived. In other embodiments, an active cosmetic ingredient is made synthetically. In some embodiments, compositions of the present technology comprise more than one active cosmetic ingredient and may include a mix of plant-derived, animal-derived and/or synthetic ingredients.

In some embodiments, the active cosmetic ingredient useful in the compositions of the present technology is tocopherol (vitamin E). Tocopherol is a fat-soluble vitamin and a natural antioxidant which can be isolated from vegetable oil. When isolated, tocopherol is a viscous oil that varies in color from yellow to brownish red. Rather than tocopherol itself, esters of tocopherol are often used in cosmetic and personal care products. These esters include, without limitation: Tocopheryl Acetate, the acetic acid ester of Tocopherol; Tocopheryl Linoleate, the linoleic acid ester of Tocopherol; Tocopheryl Linoleate/Oleate, a mixture of linoleic and oleic acid esters of Tocopherol; Tocopheryl Nicotinate, the nicotinic acid ester of Tocopherol; and Tocopheryl Succinate, the succinic acid ester of Tocopherol. Potassium Ascorbyl Tocopheryl Phosphate, a salt of both vitamin E (Tocopherol) and vitamin C (Ascorbic Acid), may also be used in compositions of the present technology. Other Tocopherol-derived ingredients that may be used include Dioleyl Tocopheryl Methylsilanol, which is the dioleyl ether of Tocopheryl Acetate monoether with methylsilanetriol, and Tocophersolan, which is also called Tocopheryl Polyethylene Glycol 1000 Succinate. The addition of succinic acid and an average of 22 ethylene oxide groups to Tocopheryl makes Tocophersolan, a water-soluble form of Tocopherol. Tocopherols can be produced from vegetable oils or can be synthesized. In addition to their antioxidant properties, tocopherols can also function as skin-conditioning agents. In some such embodiments, the composition of the present technology thus comprises CBD or a derivative thereof; and tocopherol or a derivative thereof.

In some embodiments, tocopherol is used to stabilize the compositions of the present technology, for example to prevent discoloration thereof and therefore enhance the shelf life of a composition. It should be understood that in some cases an active cosmetic ingredient such as tocopherol may have both therapeutic or cosmetic properties as well as non-medicinal properties. Similarly, some non-medicinal cosmetic ingredients may also provide active therapeutic or cosmetic benefits in certain cases. The term “active cosmetic ingredient” or “non-medicinal cosmetic ingredient” may therefore depend on the context of the ingredient in the composition, such as its concentration, presence or absence of other ingredients, and the like, as will be understood by the person skilled in the art of such formulations.

In some embodiments, the active cosmetic ingredient useful in the compositions of the present technology is ubiquinone/ubiquinol (Coenzyme Q10). Uniquinone is a naturally-occurring antioxidant that plays an important role in energy production. It is used in cosmetics and personal care products such as skin care products for its anti-aging, anti-wrinkle, and energizing properties, e.g, to smooth, brighten and increase skin radiance. In some such embodiments, the composition of the present technology thus comprises CBD or a derivative thereof; and ubiquinone or a derivative thereof.

In some embodiments, the active cosmetic ingredient useful in the compositions of the present technology is Caprylic/Capric Triglyceride. Caprylic/Capric Triglyceride is made from coconut oil and/or coconut acid (which is a mixture of fatty acids derived from coconut oil). It is often used in cosmetic and personal care products as a skin conditioning agent or an emollient for protecting, moisturizing and/or lubricating the skin. In some such embodiments, the composition of the present technology thus comprises CBD or a derivative thereof; and Caprylic/Capric Triglyceride or a derivative thereof.

In some embodiments, the active cosmetic ingredient useful in the compositions of the present technology is Squalane. Squalane and Squalene are oily substances that are common components of other oils. Both Squalane and Squalene are natural components of human sebum, a mixture of lipids produced by glands in the skin. Squalene is found in large quantities in shark liver oil, and in smaller amounts in olive oil, wheat germ oil, rice bran oil, yeast, and in various other foodstuffs. Squalane is made by completely hydrogenating Squalene. The hydrogenation process can occur naturally in the human body. Squalane and Squalene act as lubricants on the skin surface, which gives the skin a soft, smooth appearance. These ingredients also act as hair conditioning agents. In cosmetics and personal care products, Squalane and Squalene are used in the formulation of a wide variety of products including bath oils, hair products, eye makeup, makeup foundations, lipstick, suntan and sunscreen products, body powders, nail products and in cleansing, moisturizing, and skin care products. In some such embodiments, the composition of the present technology thus comprises CBD or a derivative thereof; and Squalane or a derivative thereof.

In some embodiments, the active cosmetic ingredient useful in the compositions of the present technology is Citric acid. Citric acid is a weak acid that is widely found in both plants and animals It is a natural ingredient that is common in citrus fruits, for example, lemon juice, which contains about 5-8 percent citric acid. Products containing citric acid and some of its salts and esters can be safely formulated for specific application to baby skin or use near the eye area or on mucous membranes. In addition, they may be used in cosmetic sprays, including hair, deodorant, body and other propellant and pump spray products. Citric acid is also one of a group of ingredients known as alpha hydroxy acids that are used as the active ingredients in chemical skin peels. Sodium citrate may be used in all types of cosmetic products, including baby products, make-up, lipstick, bath products, soaps and detergents, hair dyes and colors, and hair and skin care products. Tributyl citrate and triethyl citrate may be used in bath products, other cleansing products, and creams and lotions. In addition, citric acid and its diammonium, potassium and sodium salts are all used to help preserve cosmetics and personal care products by chelating metals. Citric acid and its salts are also added to cosmetics to help adjust the acid/base balance. The citric acid ingredients most commonly used in cosmetics are citric acid, sodium citrate, tributyl citrate and triethyl citrate. In some such embodiments, the composition of the present technology thus comprises CBD or a derivative thereof; and Citric acid or a salt or a derivative thereof.

In some embodiments, the active cosmetic ingredient useful in the compositions of the present technology is Sodium hyaluronate. Hyaluronic Acid is made up of many sugar units. With the addition of more sugar molecules, it grows longer and the overall shape becomes spherical. It is normally found in skin, connective and nerve tissue. Compared to other natural and synthetic polymers, Hyaluronic Acid has the greatest capacity to hold water. Sodium Hyaluronate and Potassium Hyaluronate are salts of Hyaluronic Acid. Hyaluronic Acid, Sodium Hyaluronate and Potassium Hyaluronate enhance the appearance of dry or damaged skin by reducing flaking and restoring suppleness. Hyaluronic Acid also has a number of approved medical uses. It has been used in eye surgery and in the treatment of osteoarthritis of the knee. It is also approved to be injected under the skin to temporarily smooth wrinkles. Hyaluronic Acid is also used to thicken the aqueous (water) portion of cosmetics and personal care products. In some such embodiments, the composition of the present technology thus comprises CBD or a derivative thereof; and Sodium hyaluronate or a derivative thereof.

In some embodiments, the active cosmetic ingredient useful in the compositions of the present technology is Parfum (fragrance). According to the U.S. Food and Drug Regulation, a fragrance is “any natural or synthetic substance or substances used solely to impart an odor to a cosmetic product.” Fragrances are complex combinations of natural and/or man-made substances that are added to many consumer products to give them a distinctive smell. Fragrances are used in a wide variety of products to impart a pleasant odor, mask the inherent smell of some ingredients, and/or enhance the experience of using the product. Fragrances can also alleviate stress and create a feeling of well-being. In some such embodiments, the composition of the present technology thus comprises CBD or a derivative thereof, and parfum (fragrance).

In some embodiments, the active cosmetic ingredient useful in the compositions of the present technology is Ascorbyl Glucoside. Ascorbyl Glucoside is a water-soluble derivative of Vitamin C combined with glucose. When absorbed into skin, it breaks down to ascorbic acid (vitamin C). It acts as a whitening agent and an antioxidant and is used as an anti-aging and skin smoothing or brightening agent. It can stimulate collagen production, reduce fine lines and wrinkles, and provide sun damage protection and repair. In some such embodiments, the composition of the present technology thus comprises CBD or a derivative thereof; and Ascorbyl Glucoside.

In some embodiments, the active cosmetic ingredient useful in the compositions of the present technology is an amino acid. The twenty most common amino acids found in proteins are: Alanine, Arginine, Asparagine, Aspartic Acid, Cysteine, Glutamic Acid, Glutamine, Glycine, Histidine, Isoleucine, Leucine, Lysine, Methionine, Phenylalanine, Proline, Serine, Threonine, Tryptophan, Tyrosine and Valine. Amino acids and simple salts of amino acids, such as Arginine HCl, Calcium Aspartate, Calcium Glycinate, Cysteine HCl, Dipotassium Aspartate, Histidine HCl, Lysine HCl, Magnesium Aspartate, Magnesium Glycinate, Potassium Aspartate, Sodium Aspartate, Sodium Glutamate and Sodium Glycinate are often used in cosmetics and personal care products such as makeup and hair and skin care products, where they can function primarily as conditioning agents. In some such embodiments, the composition of the present technology thus comprises CBD or a derivative thereof, and one or more amino acid. In some such embodiments, the one or more amino acid is Arginine, Aspartic Acid, Glycine, Alanine, Serine, Valine, Isoleucine, Proline, Threonine, Histidine, Phenylalanine, or a combination thereof.

In some embodiments, the active cosmetic ingredient useful in the compositions of the present technology is a retinoid such as retinol or hydroxypinacolone retinoate (HPR; also known as “Granactive Retinoid”). Retinol is the primary naturally occurring form of vitamin A. HPR is a retinoic acid ester and binds directly to retinoid receptors of skin cells. Retinoids/retinol products are made from vitamin A and are widely used for their powerful anti-aging, anti-wrinkle, and anti-acne properties. They reduce fine lines and wrinkles by stimulating production of collagen, and improve skin texture, pigmentation, and tone. Other examples of retinoids that may be used in compositions of the present technology include, without limitation, tretinoin (Retin-A®), tazarotene, and adapalene. In some such embodiments, the composition of the present technology thus comprises CBD or a derivative thereof; and a retinoid/retinol product. In some such embodiments, the composition of the present technology thus comprises CBD or a derivative thereof, and retinol or hydroxypinacolone retinoate (HPR).

In some embodiments, the active cosmetic ingredient useful in the compositions of the present technology is glycolic acid, lactic acid and/or sodium lactate. Glycolic Acid and Lactic Acid are naturally occurring organic acids also known as Alpha Hydroxy Acids or AHAs. The salts of Glycolic Acid (Ammonium Glycolate, Sodium Glycolate), the salts of Lactic Acid (Ammonium Lactate, Calcium Lactate, Potassiu Lactate, Sodium Lactate, TEA-Lactate) and the esters of Lactic Acid (Methyl Lactate, Ethyl Lactate, Butyl Lactate, Lauryl Lactate, Myristyl Lactate, Cetyl Lactate) may also be used in cosmetics and personal care products. In cosmetics and personal care products, these ingredients are used in the formulation of moisturizers, cleansing products, and other skin care products. Glycolic acid, Lactic acid and Sodium lactate are commonly used as exfoliants, skin conditioning agents or buffering agents. In some such embodiments, the composition of the present technology thus comprises CBD or a derivative thereof; and glycolic acid, lactic acid and/or sodium lactate.

In some embodiments, the at least one active cosmetic ingredient useful in the compositions of the present technology is bakuchiol. Bakuchiol is mainly obtained from the seeds of the plant Psoralea corylifolia and can also be made synthetically. It is marketed under the trade name Sytenol® A. Bakuchiol has antioxidant, anti-inflammatory and anti-bacterial properties. It also has retinol functionality through retinol-like regulation of gene expression and has been shown to be comparable with retinol clinically in its ability to improve photo-aging (wrinkles, hyperpigmentation). Bakuchiol is sometimes referred to as a “retinol alternative”. In some such embodiments, the composition of the present technology thus comprises CBD or a derivative thereof; and bakuchiol or a derivative thereof.

In some embodiments, the active cosmetic ingredient useful in the compositions of the present technology is a complex of alpha hydroxy acids (AHAs). The AHAs most commonly used in cosmetic products are glycolic acid (which is derived from sugar cane) and lactic acid. Other AHAs used include citric acid (from oranges, lemons, etc.), 2-hydroxyoctanoic acid, 2-hydroxydecanoic acid, and tartaric acid. The AHA's may be obtained from their natural sources or may be made synthetically. AHAs may be used in cosmetics and personal care products as discussed above and to exfoliate skin, reduce signs of aging, and improve skin clarity, complexion, texture and tone. AHAs generally work by promoting cell shedding in the outer layers of the epidermis and by restoring hydration. In some such embodiments, the composition of the present technology thus comprises CBD or a derivative thereof; and one or more AHA or a derivative or mixture thereof.

In some embodiments, the active cosmetic ingredient useful in the compositions of the present technology is Glycerin. Glycerin (glycerol) is a naturally occurring alcohol compound found in all animal and plant tissues. It can be obtained from natural sources or made synthetically. It is used in cosmetics and personal care products such as skin care products to provide smoothness and lubrication, as a skin conditioning agent, and as a skin protectant. In some such embodiments, the composition of the present technology thus comprises CBD or a derivative thereof; and glycerin.

In some embodiments, the active cosmetic ingredient useful in the compositions of the present technology is Sodium stearoyl glutamate. Sodium stearoyl glutamate is a synthetic or natural (animal-derived) ingredient that is added to cosmetics and personal care products to condition skin or work as an emulsifier. In some such embodiments, the composition of the present technology thus comprises CBD or a derivative thereof; and Sodium stearoyl glutamate.

In some embodiments, the active cosmetic ingredient useful in the compositions of the present technology is Aloe barbadensis Leaf Juice Powder. Aloe barbadensis Flower Extract, Aloe barbadensis Leaf, Aloe barbadensis Leaf Extract, Aloe barbadensis Leaf Juice, Aloe barbadensis Leaf Polysaccharides and Aloe barbadensis Leaf water are ingredients made from the plant Aloe barbadensis, which is also commonly called Aloe vera. Other species of aloe, such as Aloe andongensis, Aloe arborescens and Aloe ferox may also be used to derive active cosmetic ingredients. These ingredients include Aloe andongensis Extract, Aloe andongensis Leaf Juice, Aloe arborescens Leaf Extract, Aloe arborescens Leaf Protoplasts, Aloe ferox Leaf Extract, Aloe ferox Leaf Juice and Aloe ferox Leaf Juice Extract. Aloe-derived ingredients are in skin care products and in makeup to enhance the appearance of dry or damaged skin by reducing flaking and restoring suppleness. Aloe is also believed to improve wound healing and reduce skin inflammation. In some such embodiments, the composition of the present technology thus comprises CBD or a derivative thereof; and an aloe-derived ingredient. In some such embodiments, the composition of the present technology thus comprises CBD or a derivative thereof; and Aloe barbadensis Leaf Juice Powder.

In some embodiments, the active cosmetic ingredient useful in the compositions of the present technology is Prunus amygdalus Dulcis (Sweet Almond) Oil. Prunus amygdalus dulcis (Sweet Almond) Oil acts as a lubricant on the skin surface, which gives skin a soft and smooth appearance. It is used in a wide variety of cosmetics and personal care products including skin care products and moisturizers. In some such embodiments, the composition of the present technology thus comprises CBD or a derivative thereof; and Prunus amygdalus dulcis (Sweet Almond) Oil.

In some embodiments, the active cosmetic ingredient useful in the compositions of the present technology is Simmondsia chinensis (Jojoba) Seed Oil Simmondsia chinensis (Jojoba) Seed Oil and Simmondsia chinensis (Jojoba) Seed Wax, also called Jojoba Oil and Jojoba Wax, are natural ingredients derived from the seeds of the desert shrub, Simmondsia chinensis. Other ingredients made from Jojoba Oil include Simmondsia chinensis (Jojoba) Butter, Hydrogenated Jojoba Oil, Jojoba Esters, Hydrolyzed Jojoba Esters, Isomerized Jojoba Oil and Jojoba Alcohol. Synthetic Jojoba Oil is a mixture of wax esters of fatty acids and alcohols that is indistinguishable from natural jojoba oil with regard to chemical composition and physical characteristics. Among the active cosmetic ingredients made from jojoba, Simmondsia chinensis (Jojoba) Seed Oil is most frequently used in cosmetics and personal care products including makeup and skin care products as a skin-conditioning agent. In some such embodiments, the composition of the present technology thus comprises CBD or a derivative thereof; and Simmondsia chinensis (Jojoba) Seed Oil.

In some embodiments, the active cosmetic ingredient useful in the compositions of the present technology is Glyceryl Stearate. Glyceryl Stearate is an esterification product of glycerin and stearic acid. It acts as a lubricant on the skin's surface, which gives the skin a soft and smooth appearance, and also forms a barrier on the skin's surface that decreases moisture loss. It can also be used as an emulsifier. In some such embodiments, the composition of the present technology thus comprises CBD or a derivative thereof; and Glyceryl Stearate.

In some embodiments, the active cosmetic ingredient useful in the compositions of the present technology is PCA or Sodium PCA. PCA (pyrrolidonecarboxylic acid) and Sodium PCA (the sodium salt of PCA) increase the water content of the top layers of the skin by drawing moisture from the surrounding air. They also enhance the appearance and feel of hair, by increasing hair body, suppleness, or sheen, or by improving the texture of hair that has been damaged physically or by chemical treatment. PCA occurs naturally in mammalian tissues, including skin. In some such embodiments, the composition of the present technology thus comprises CBD or a derivative thereof; and PCA or Sodium PCA.

In some embodiments, the active cosmetic ingredient useful in the compositions of the present technology is ethylhexylglycerin. Ethylhexylglycerin is an alkyl glyceryl ether, having an ethylhexyl group bound to glycerin at one end by an ether linkage. Ethylhexylglycerin and other alkyl glyceryl ether ingredients are used in a wide range of cosmetics and personal care products as skin conditioning agents, deodorant agents, and/or preservative-enhancing agents. In some such embodiments, the composition of the present technology thus comprises CBD or a derivative thereof; and ethylhexylglycerin.

In some embodiments, the active cosmetic ingredient useful in the compositions of the present technology is Lecithin. Lecithin is a naturally occurring mixture of the diglycerides of stearic, palmitic and oleic acids, linked to the choline ester of phosphoric acid whose form varies from a waxy mass to a thick, pourable liquid. Hydrogenated Lecithin is the product of controlled hydrogenation (addition of hydrogen) of Lecithin. Lecithin and Hydrogenated Lecithin are used in the formulation of a large number of cosmetics and personal care products to enhance the appearance of dry or damaged skin by reducing flaking and restoring suppleness. These ingredients also help to form emulsions. In some such embodiments, the composition of the present technology thus comprises CBD or a derivative thereof, and Lecithin.

In some embodiments, the active cosmetic ingredient useful in the compositions of the present technology is BHA and/or BHT. BHA (Butylated Hydroxyanisole) and BHT (Butylated Hydroxtoluene) are used in a wide range of cosmetic and personal care products for their antioxidant properties. In some such embodiments, the composition of the present technology thus comprises CBD or a derivative thereof; and BHA and/or BHT.

In some embodiments, the active cosmetic ingredient useful in the compositions of the present technology is an Essential Oil. An essential oil is a concentrated hydrophobic liquid containing volatile chemical compounds from plants. Essential oils are also known as volatile oils, ethereal oils, aetherolea, or simply as the oil of the plant from which they were extracted, such as oil of clove. Essential oils generally contain volatile chemical compounds that give plants their characteristic odors. Essential oils are generally extracted by distillation, often by using steam. Other processes include expression, solvent extraction, sfumatura, absolute oil extraction, resin tapping, wax embedding, and cold pressing. They are used in perfumes, cosmetics, soaps and other products. In addition, essential oils are often used for aromatherapy. Examples of essential oils that may be used in compositions of the present technology include, without limitation: Vitis vinifera Seed (Grapeseed) Oil; Lavandula angustifolia (Lavender) Oil; Cinnamomum camphora Leaf (Ho Wood) Oil; Pogostemon cablin (Patchouli) Oil; Vetiveria zizanoides Root (Vetiver) Oil; Chamaemelum nobile Flower (Roman Chamomile) Oil; Cocos nucifera (Coconut) Oil; Prunus amygdalus Dulcis (Sweet Almond) Oil; and Simmondsia chinensis (Jojoba) Seed Oil. In some such embodiments, the composition of the present technology thus comprises CBD or a derivative thereof, and one or more essential oil selected from Vitis vinifera Seed (Grapeseed) Oil; Lavandula angustifolia (Lavender) Oil; Cinnamomum camphora Leaf (Ho Wood) Oil; Pogostemon cablin (Patchouli) Oil; Vetiveria zizanoides Root (Vetiver) Oil; Chamaemelum nobile Flower (Roman Chamomile) Oil; Cocos nucifera (Coconut) Oil; Prunus amygdalus dulcis (Sweet Almond) Oil; Simmondsia chinensis (Jojoba) Seed Oil; and combinations thereof.

Other non-limiting examples of essential oils that may be used in compositions of the present technology include orange, lavender, peppermint, thyme, patchouli, oregano, lemon, grapefruit, bergamot, tea tree, ho wood, eucalyptus, mugwort, pennyroyal, chamomile, wormwood, marjoram, black pepper, clary sage, sage, clove, rosemary, cinnamon, lemongrass, ylang ylang, sandalwood, jasmine, rose, vetivert, frankincense, myrrh, helichrysum, cedarwood, spearmint, basil, and the like. In some embodiments, lavender, vetivert and chamomile are used in compositions of the present technology. In other embodiments, lavender, vetivert, chamomile and patchouli are used. In still other embodiments, lavender, vetivert, chamomile, patchouli and ho wood are used. In some embodiments, marjoram, black pepper and clove oils are used. In further embodiments, lavender oil, vetivert oil, chamomile oil, patchouli oil, ho wood oil, grapeseed oil, coconut oil, and combinations thereof are used.

In an embodiment, the active cosmetic ingredient useful in the compositions of the present technology comprises Lavender oil. Lavender oil is an essential oil obtained by distillation from the flower spikes of certain species of lavender. Lavender oil has long been used as a perfume, for aromatherapy, and for skin applications for its relaxation, calming, anxiolytic, soothing and sedative effects. In some such embodiments, the composition of the present technology thus comprises CBD or a derivative thereof; and lavender oil, or an extract thereof.

In an embodiment, the active cosmetic ingredient useful in the compositions of the present technology comprises vetivert oil. Vetivert oil (Vetiver Essential Oil) is derived from the aromatic roots of the Vetiveria zizanioides (Linn) Nash. botanical. Due to its grounding, sensuous, and deeply calming scent, vetiver essential oil is also referred to as the “Oil of Tranquility” and “The Fragrance of the Soil.” Used in aromatherapy applications, vetiver essential oil is beneficial for soothing anxiety, insomnia, fatigue, depression, and inability to concentrate or remember. In some such embodiments, the composition of the present technology thus comprises CBD or a derivative thereof, and vetivert oil, or an extract thereof.

In an embodiment, the active cosmetic ingredient useful in the compositions of the present technology comprises chamomile oil. Chamomile essential oil has powerful anti-inflammatory and calming properties. The main benefits of chamomile oil are its ability to calm nerves, fight anxiety and depression. Its calming compounds serve as an emotional trigger to help reduce anxiety, boost mood and eliminate stress. In some such embodiments, the composition of the present technology thus comprises CBD or a derivative thereof, and chamomile oil, or an extract thereof.

In some embodiments, the active cosmetic ingredient useful in the compositions of the present technology comprises patchouli oil. Patchouli oil is derived from the leaves of the highly-valued Patchouli plant, which belongs to a family of other well-known aromatic plants, including Lavender, Mint, and Sage. It has a warm, spicy, musky and sensuous scent. It has a grounding, balancing, calming scent and provides numerous health benefits, making it ideal for therapeutic use in cosmetics and aromatherapy. In some such embodiments, the composition of the present technology thus comprises CBD or a derivative thereof, and patchouli oil, or an extract thereof.

In some embodiments, the active cosmetic ingredient useful in the compositions of the present technology comprises ho wood oil. Ho Wood Oil is extracted from the leaves of Cinnamomum camphora. It is extremely high in calming linalool and is excellent for promoting a peaceful and relaxing environment. Ho Wood has a woody, floral, and camphorous scent. In some such embodiments, the composition of the present technology thus comprises CBD or a derivative thereof, and Ho wood oil, or an extract thereof.

In some embodiments, the active cosmetic ingredient useful in the compositions of the present technology comprises grapeseed oil. Grapeseed oil comes from the pressed seeds of grapes. The oil is a by-product of the process of making wine. It is known for its anti-inflammatory, antimicrobial and antioxidant properties. These properties, along with the high amounts of omega chain fatty acids and vitamin E contained in grapeseed oil, have made it a popular topical treatment for skin. In some such embodiments, the composition of the present technology thus comprises CBD or a derivative thereof, and grapeseed oil, or an extract thereof.

In some embodiments, the active cosmetic ingredient useful in the compositions of the present technology comprises coconut oil. Coconut oil, or copra oil, is an edible oil extracted from the kernel or meat of mature coconuts harvested from the coconut palm. It has various applications. Coconut Essential Oil is commonly used in aromatherapy for alleviating a number of skin issues, including eczema, psoriasis, dermatitis, and dandruff. It is known to reduce damaging inflammation and moisturize the skin to allow quicker healing. In some such embodiments, the composition of the present technology thus comprises CBD or a derivative thereof, and coconut oil, or an extract thereof.

In some embodiments, the active cosmetic ingredient useful in the compositions of the present technology comprises a vitamin. In some implementations of these embodiments, the vitamin is one or more of: vitamin A (as all-trans-retinol, all-trans-retinyl-esters, as well as all-trans-beta-carotene and other provitamin A carotenoids), vitamin B1 (thiamine), vitamin B2 (riboflavin), vitamin B3 (niacin), vitamin B5 (pantothenic acid), vitamin B6 (pyridoxine), vitamin B7 (biotin), vitamin B9 (folic acid or folate), vitamin B12 (cobalamins), vitamin C (ascorbic acid), vitamin D (calciferols), vitamin E (tocopherols and tocotrienols), and vitamin K (quinones). In some further implementations, the vitamin is Vitamin A (retinol). Vitamins may be present in the compositions of the present disclosure in an amount ranging from between about 0.001 wt % and about 2 wt %, or between about 0.01 wt % and about 1 wt %, or between about 0.05 wt % and about 2 wt %, or between about 0.05 wt % and about 1 wt %. In some such embodiments, the composition of the present technology thus comprises CBD or a derivative thereof, and one or more vitamin or a derivative thereof.

In some embodiments, the active cosmetic ingredient useful in the compositions of the present technology comprises a functional fragrance. As used herein, the term “functional fragrance” refers to a compound that activates olfactory receptors leading to activation of neural circuitry in the brain promoting psychological or physical well-being. A functional fragrance may, for example, provide improved sleep, relaxation, calm, and/or reduced anxiety. A functional fragrance may include, for example and without limitation, one or more essential oil; one or more purified terpene; or one or more essential oil and one or more terpene. A functional fragrance may include odorants extracted or isolated from natural sources, synthetic odorants, and combinations thereof. It should be understood that a wide range of odorants may be used in functional fragrances in the compositions of the present technology. In some such embodiments, the composition of the present technology thus comprises CBD or a derivative thereof, and one or more functional fragrance, or an extract or derivative thereof.

In some embodiments, the functional fragrance useful in the compositions of the present technology comprises between about 0.005 wt % and about 10 wt %, or between about 0.05 wt % and about 10 wt %, or between about 0.05 wt % and about 5 wt %, or between about 0.1 wt % and about 10 wt %, or between about 0.1 wt % and about 5 wt % of one or more essential oil disclosed herein.

In some embodiments, the functional fragrance useful in the compositions of the present technology comprises at least one purified terpene and/or terpenoid. Terpenes and terpenoids are the primary constituents of the essential oils or many types of plants and flowers. Terpenes can be converted to a terpenoid, synthetic terpenoid or semisynthetic terpenoid by any known chemical reactions. In some embodiments, the terpenes include, for example, alpha-Pinene, beta-Pinene, beta-Myrcene, alpha-Terpinene, Limonene, beta-Ocimene, Terpinolene, Linalool, Fenchyl Alcohol, Borneol Isomers, Alpha-Terpineol, Trans-caryophyllene, Alpha-humulene, Trans-nerolidol, Guaiol, Alpha-Bisabolol, and combinations thereof. Suitable terpenoids (and substances that include combinations of terpenes and terpenoids) include α-Pinene, β-Pinene, pine, linalool, linalool oxide, linalyl acetate, lavender, black pepper, myrcene, musk, limonene, citrus, terpineol, lilac, geraniol, citronellol, eugenol, citral, farnesol, bakuchiol, nerolidol, wood bark, eucalyptol, mint, borneol, camphor; α-bisabolol, floral; D-3 Carene, pine, camphene, herbal, β-caryophyllene, Borneol, 1,8-cineole, camphene, humulene, limonene, nerolidol, pulegone, terpinolene, α-phellandrene, Δ3-carene, α-terpinene, β-phellandrene, cis-ocimene, terpinolene, β-caryophyllene, α-guaiene, humulene, δ-guaiene, elemene, guaiol, γ-eudesmol, β-eudesmol, agarospirol, bulnesol, and α-bisabolol. In one embodiment, the at least one purified terpene is selected from linalool, linalool oxide, limonene, geraniol, citronellol, eugenol, citral, farnesol, bakuchiol, derivatives thereof, and combinations thereof. In some such embodiments, the composition of the present technology thus comprises CBD or a derivative thereof, and one or more functional fragrance comprising at least one purified terpene or terpenoid. In some such embodiments, the composition of the present technology thus comprises CBD or a derivative thereof, and a functional fragrance comprising one or more of Linalool, Linalyl Acetate, Vetiverols, Ocimene, Caryophyllene, Terpentine-4-ol, beta-Pinene, d-Limonene, 1-Octen-3-yl acetate, Geraniol, derivatives thereof, and combinations thereof.

In some embodiments, the functional fragrance useful in the compositions of the present technology comprises linalool. Linalool refers to two enantiomers (S-(+)-linalool and R-(—)-linalool) of a naturally occurring terpene alcohol found in many flowers and spice plants. Its characteristic lavender scent with a hint of spiciness is common to over 200 types of plants. These have multiple commercial applications, the majority of which are based on its pleasant scent. Linalool has sedative, anxiolytic, and anticonvulsant properties (Russo, E. B., Br J Pharmacol. 163(7):1344-64, 2011) and may also assist in pain management. Notably, S-(+)-linalool, and R-(−)-linalool have been shown to be as effective as the trazodone hydrochloride in reducing anxiety. Although it is not clear exactly how linalool exerts its effects on the brain, it appears not to act directly on cannabinoid receptors, and may instead act to block excitatory glutamate receptors and/or inhibit acetylcholine signaling Linalool falls under the Generally-recognized-as-safe (GRAS) classification, and has been shown to be safe at doses as high as 120 mg. In some such embodiments, the composition of the present technology thus comprises CBD; and linalool, linalool oxide, or a derivative thereof.

In some embodiments, the functional fragrance useful in the compositions of the present technology comprises limonene. Limonene is a cyclic monoterpene and is the major component in the oil of citrus fruit peels. D-Limonene is a major component of the aromatic scents and resins characteristic for numerous coniferous and broadleaved trees, such as cottonwoods, aspens, spruces, and various pines, firs and cedars. It contributes to the characteristic odor of orange peel, orange juice and other citrus fruits. The D-isomer, occurring more commonly in nature as the fragrance of oranges, is commonly used as a flavoring agent in food manufacturing and as a fragrance in cosmetics and personal care products. D-limonene is the second most widely distributed terpenoid in nature. It has been shown to have anxiolytic and anti-depression activity. It has also been used to make medicines for treating obesity, bronchitis, and cancer, although these effects are not proven. It is also added to medicinal ointments and creams to facilitate skin penetration. It is nontoxic and highly bioavailable. In some such embodiments, the composition of the present technology thus comprises CBD; and limonene or a derivative thereof.

In some embodiments, the functional fragrance useful in the compositions of the present technology comprises geraniol. Geraniol is a monoterpenoid and is the primary component of rose oil, palmarosa oil, and citronella oil. It also occurs in small quantities in geranium, lemon, and many other essential oils. It is used as an agent for flavoring and fragrance in foods and cosmetics. It is also commonly used as an insect repellent. It is closely related structurally to citral and to linalool, which is an isomer. In some such embodiments, the composition of the present technology thus comprises CBD; and geraniol or a derivative thereof.

In some embodiments, the functional fragrance useful in the compositions of the present technology comprises vetiverol. Vetiverol (also known as vetivenol) is a mixture of sesquiterpenoid alcohols obtained from vetiver oil and often used in perfumes. It is inhaled as aromatherapy for nervousness, insomnia, and joint and muscle pain. It is also applied directly to the skin for relieving stress, as well as for emotional traumas and shock, for stings and burns, for arthritis, and to repel insects. In some such embodiments, the composition of the present technology thus comprises CBD; and vetiverol or a derivative thereof.

In some embodiments, the functional fragrance useful in the compositions of the present technology comprises Linalyl Acetate. Linalyl acetate is a naturally-occurring phytochemical found in many flowers and spice plants. It is one of the principal components of the essential oils of bergamot and lavender. Chemically, it is the acetate ester of linalool, and the two often occur in conjunction. It is commonly used in aromatherapy and massage therapy due to its ability to promote relaxation and better mood when inhaled. In some such embodiments, the composition of the present technology thus comprises CBD; and linalyl acetate or a derivative thereof.

In some embodiments, the functional fragrance useful in the compositions of the present technology comprises ocimene. Ocimene is a cannabis terpene carrying a sweet, herbaceous, woody and earthy aroma with hints of citrus. Also found in mint, parsley, basil, mangoes, orchids, oregano and tarragon, ocimene is commonly used in perfumes and fragrances for its sweet and floral aromatic profile. This terpene contributes significantly to the floral odor of various plant species, including the scent of orchid and snapdragon flowers. Ocimene is also a pheromone involved in social regulation in the honey bee colony. It is believed to have many medicinal effects including antifungal, antiviral, and anti-inflammatory effects. In some such embodiments, the composition of the present technology thus comprises CBD; and ocimene or a derivative thereof.

In some embodiments, the functional fragrance useful in the compositions of the present technology comprises Caryophyllene. Caryophyllene is present in many herbs and spices, including black pepper, basil, and oregano, and cannabis. It is a natural bicyclic sesquiterpene that is a constituent of many essential oils, especially clove oil, the oil from the stems and flowers of Syzygium aromaticum, and the essential oil of Cannabis sativa, rosemary, and hops. It is widely considered to provide digestive protection, pain relief, and act as an antibacterial agent. In some such embodiments, the composition of the present technology thus comprises CBD; and caryophyllene or a derivative thereof.

In some embodiments, the functional fragrance useful in the compositions of the present technology comprises Terpentine-4-ol. Terpentine-4-ol is an isomer of terpineol. A primary constituent of tea tree oil, it is obtained as an extract from the leaves, branches, and bark of Melaleuca alternifolia Cheel. In some such embodiments, the composition of the present technology thus comprises CBD; and Terpentine-4-ol or a derivative thereof.

In some embodiments, the functional fragrance useful in the compositions of the present technology comprises beta-Pinene. beta-Pinene has a woody-green pine-like smell and is one of the most abundant compounds released by forest trees. It is useful for relieving pain, inflammation and anxiety. In some such embodiments, the composition of the present technology thus comprises CBD; and beta-Pinene or a derivative thereof.

In some embodiments, the functional fragrance useful in the compositions of the present technology comprises 1-Octen-3-yl acetate. 1-Octen-3-yl acetate is a component of lavender oil. In some such embodiments, the composition of the present technology thus comprises CBD; and 1-Octen-3-yl acetate or a derivative thereof.

In some embodiments, the functional fragrance useful in the compositions of the present technology comprises eugenol. Eugenol is an aromatic oily liquid extracted from certain essential oils such as clove oil, nutmeg, cinnamon, basil, and bay leaf. It has a pleasant, spicy, clove-like scent. Eugenol is commonly used in perfumes, flavorings, and essential oils. It is also used as a local antiseptic and anesthetic. In some such embodiments, the composition of the present technology thus comprises CBD or a derivative thereof; and eugenol or a derivative thereof.

In some embodiments, the functional fragrance useful in the compositions of the present technology is citral. Citral is found in the oils of several plants, including lemon myrtle, lemongrass, lemon verbena, lemon balm, lime, lemon, and orange. It has a strong lemon or citrus odor and is therefore used as a fragrance and as a flavoring agent. It also has strong antimicrobial properties. In some such embodiments, the composition of the present technology thus comprises CBD or a derivative thereof; and citral or a derivative thereof.

In some embodiments, the functional fragrance useful in the compositions of the present technology is citronellol. Citronellol is an acyclic monoterpenoid found in geranium, rose, and other plants, as well as citronella oils. It has a grassy, citrus-like scent. It is commonly used as a fragrance in cosmetic and personal care products and is a good insect repellant (particularly for mosquitoes). It is generally regarded as safe (GRAS) for food use. In some such embodiments, the composition of the present technology thus comprises CBD or a derivative thereof; and citronellol or a derivative thereof.

In some embodiments, the functional fragrance useful in the compositions of the present technology is farnesol. Farnesol is an acyclic sesquiterpene alcohol. It is present in many essential oils such as citronella, neroli, cyclamen, lemon grass, tuberose, rose, musk, balsam and tolu. It is used in perfumery to emphasize the odors of sweet floral perfumes, where it can act as a co-solvent that regulates the volatility of the odorants. It is especially used in lilac perfumes. In some such embodiments, the composition of the present technology thus comprises CBD or a derivative thereof; and farnesol or a derivative thereof.

In some embodiments, the composition of the present technology comprises one or more of: Cinnamomum camphora (camphor) leaf oil; Lavandula angustifolia (lavender) oil; Ormenis multicaulis (chamomile) flower extract; Pogostemon cablin (patchouli) oil; Vetiveria zizanoides (vetivert) root oil; and Zingiber officinale (ginger) root extract.

In some embodiments, the composition of the present technology comprises one or more of: Boswellia carterii (frankincense) gum oil; Cinnamomum camphora (camphor) oil; citrus Aurantium amara (bitter orange) leaf oil; Cymbopogon martini (palmarosa) oil; Eucalyptus globulus leaf oil; and Pogostemon cablin (patchouli) oil.

In some embodiments, the composition of the present technology further comprises THC. In some embodiments, the composition of the present technology comprises a mixture of CBD and THC in a ratio of 100:1.

The compositions of the present disclosure may include the at least one purified cannabinoid in an amount ranging from between about 1 mg/mL and about 250 mg/mL, or from between about 1 mg/mL and about 200 mg/mL, or from between about 1 mg/mL and about 150 mg/mL, or from between about 1 mg/mL and about 100 mg/mL, or from between about 1 mg/mL and about 75 mg/mL, or from between about 10 mg/mL and about 250 mg/mL, or from between about 10 mg/mL and about 200 mg/mL, or from between about 10 mg/mL and about 150 mg/mL, or from between about 10 mg/mL and about 100 mg/mL, or from between about 10 mg/mL and about 75 mg/mL, or from between about 25 mg/mL and about 250 mg/mL, or from between about 25 mg/mL and about 200 mg/mL, or from between about 25 mg/mL and about 150 mg/mL, or from between about 25 mg/mL and about 100 mg/mL, or from between about 25 mg/mL and about 75 mg/mL. In some embodiments, the composition comprises the at least one purified cannabinoid in an amount of about 1 mg/mL, about 5 mg/mL, about 10 mg/mL, about 50 mg/mL, about 75 mg/mL, about 100 mg/mL, or about 150 mg/mL.

The compositions of the present technology may include the at least one active cosmetic ingredient in an amount from about 0.01 wt % to about 99 wt % of the total weight of the composition; or in an amount of between about 0.01 wt % to about 50 wt %; or in an amount of between about 0.01 wt % to about 40 wt %; or in an amount of between about 0.01 wt % to about 30 wt %; or in an amount of between about 0.01 wt % to about 20 wt %; or in an amount of between about 0.01 wt % to about 10 wt %; or in an amount of between about 0.01 wt % to about 5 wt %; or in an amount of between about 0.01 wt % to about 4 wt %; or in an amount of between about 0.01 wt % to about 3 wt %; or in an amount of between about 0.01 wt % to about 2 wt %; or in an amount of between about 0.01 wt % to about 1 wt %; or in an amount of between about 1 wt % to about 5 wt %; or in an amount of between about 1 wt % to about 50 wt %; or in an amount of between about 10 wt % to about 50 wt %; or in an amount of between about 20 wt % to about 50 wt %; or in an amount of between about 30 wt % to about 50 wt %; or in an amount of between about 10 wt % to about 99 wt %; or in an amount of between about 25 wt % to about 99 wt %; or in an amount of between about 50 wt % to about 99 wt %; or in an amount of between about 75 wt % to about 99 wt %; or in an amount of about 1 wt %, about 3 wt %, about 5 wt %, or in an amount of about 10 wt %, or in an amount of about 25 wt %, or in an amount of about 50 wt %, or in an amount of about 75 wt %, or in an amount of about 90 wt %, or in an amount of about 99 wt % of the total weight of the composition.

The compositions of the present technology may include at least one purified cannabinoid and at least one active cosmetic ingredient in a weight ratio of purified cannabinoid to active cosmetic ingredient that is between about 100:1 and about 1:500, or between about 10:1 and about 1:500, or between about 5:1 and about 1:500, or between about 10:1 and about 1:100, or between about 1:1 and about 1:100, or between about 10:1 and about 1:50, or between about 1:1 and about 1:50, or between about 10:1 and about 1:20, or between about 10:1 and about 1:10, or between about 1:1 and about 1:10.

The compositions of the present technology may include at least one purified cannabinoid and at least one active cosmetic ingredient in a weight ratio of purified cannabinoid to active cosmetic ingredient that is about 100:1, about 10:1, about 5:1, about 2:1, about 1:1, about 1:2, about 1:3, about 1:4, about 1:5, about 1:6, about 1:7, about 1:8, about 1:9, about 1:10, about 1:20; about 1:25, about 1:35, about 1:50, about 1:75, about 1:100; about 1:110, about 1:125, about 1:200, or about 1:500.

The compositions of the present technology may include at least one terpene in an amount from about 0.001 mg/mL to about 1 mg/mL. In other embodiments, the composition may comprise at least one terpene in an amount of about 0.001 mg/mL to about 0.95 mg/mL, or about 0.001 mg/mL to about 0.9 mg/mL, or about 0.005 mg/mL to about 0.8 mg/mL. In some embodiments, the composition may comprise at least one terpene in an amount of about 0.01 mg/mL, about 0.15 mg/mL, about 0.02 mg/mL, about 0.25 mg/mL, about 0.03 mg/mL, about 0.35 mg/mL, about 0.04 mg/mL, about 0.45 mg/mL, about 0.05 mg/mL, about 0.55 mg/mL, about 0.06 mg/mL, about 0.65 mg/mL, about 0.07 mg/mL, about 0.75 mg/mL, about 0.08 mg/mL, about 0.085 mg/mL, about 0.09 mg/mL, about 0.95 mg/mL, or about 1 mg/mL. The concentrations listed are the total concentration of all the terpenes in the composition.

In an embodiment, the composition of the present disclosure includes one or more of THC, CBDA, CBD, CBGA, CBG, CBCA, CBC, THCVA, THCV, CBDVA, and CBDV, and one or more active cosmetic ingredient selected from: Ascorbyl Glucoside, Ubiquinone, one or more alpha hydroxy acid (AHA), Bakuchiol, Hydroxypinacolone Retinoate (HPR), and combinations thereof.

In another embodiment, the composition of the present disclosure includes one or more of THC, CBDA, CBD, CBGA, CBG, CBCA, CBC, THCVA, THCV, CBDVA, and CBDV, and one or more active cosmetic ingredient selected from: Ubiquinone, Caprylic/Capric Triglyceride, Squalane, Citric Acid, Ascorbyl Glucoside, Sodium hyaluronate, one or more amino acid, retinol, hydroxypinacolone retinoate (HPR), one or more alpha hydroxy acid (AHA), sodium lactate, glycerin, Aloe Vera, Aloe barbadensis Leaf Juice Powder, one or more essential oil, Glyceryl stearate, Octyldodecanol, PCA, Sodium PCA, ethylhexylglycerin, lecithin, BHA, BHT, derivatives thereof, and combinations thereof.

In some such embodiments, the composition of the present disclosure further comprises at least one antioxidant such as tocopherol to increase stability of the composition, e.g., by preventing discoloration or other spoilage.

In some such embodiments, the one or more AHA is selected from: glycolic acid, lactic acid, tartaric acid, and citric acid. In some such embodiments, the one or more essential oil is selected from: Vitis vinifera Seed (Grapeseed) Oil; Lavandula angustifolia (Lavender) Oil; Cinnamomum camphora Leaf (Ho Wood) Oil; Pogostemon cablin (Patchouli) Oil; Vetiveria zizanoides Root (Vetiver) Oil; Chamaemelum nobile Flower (Roman Chamomile) Oil; Cocos nucifera (Coconut) Oil; Prunus amygdalus dulcis (Sweet Almond) Oil; Simmondsia chinensis (Jojoba) Seed Oil; and combinations thereof. In some such embodiments, the one or more amino acid is selected from: Arginine, Aspartic Acid, Glycine, Alanine, Serine, Valine, Isoleucine, Proline, Threonine, Histidine, Phenylalanine, and combinations thereof.

In some embodiments of the present technology, the composition of the present disclosure comprises at least one or more of the following: Phenoxyethanol, Sodium Metabisulfite, Dimethyl Isosorbide, Tartaric Acid, Magnesium Chloride, Sodium stearoyl glutamate, Benzyl alcohol, Butylene glycol, Xanthan Gum, Polysorbate 20, Disodium EDTA, Sodium hydroxide, Dehydroacetic Acid, Stearic acid, Cetyl alcohol, Ceteth-20, Steareth-20, PEG-75 Stearate, C10-30 Alkyl Acrylate Crosspolymer, Parfum (fragrance), or derivatives thereof, or combinations thereof.

In some embodiments of the present technology, therefore, the composition of the present disclosure comprises CBD or a derivative thereof and at least one active cosmetic ingredient selected from an antioxidant, an anti-aging agent, an anti-wrinkle agent, an anti-inflammatory agent, an anti-acne agent, an exfoliant an emollient, a moisturizer, and a skin-conditioning agent.

In some embodiments of the present technology, the composition of the present disclosure comprises CBD or a derivative thereof and one or more of a retinoid or a derivative thereof and a vitamin or a derivative thereof. In some such embodiments, the composition further comprises at least one antioxidant.

In some embodiments, the compositions of the present disclosure comprise at least one purified cannabinoid and at least one antioxidant in a weight ratio of between about 1:1 and about 1:5, or between about 1:1 and about 1:10.

In some embodiments, the compositions of the present disclosure comprise at least one purified cannabinoid and at least one antioxidant in a weight ratio of between about 1:1 and about 1:5, or between about 2:1 and about 30:1, or between about 2:1 and about 20:1, or between about 5:1 and 10:1.

In some embodiments of the present technology, the composition of the present disclosure comprises CBD or a derivative thereof; an active cosmetic ingredient; and an additional botanical extract.

In some implementations of these embodiments, the additional botanical extract may include one or more of angelica extract, avocado extract, Tasmannia lanceolata extract, wild yam extract, Boswellia spp. extract, fenugreek extract, Harpagophytum spp. extract, hydrangea extract, althea extract, Arnica spp. extract, aloe extract (also referred to herein as aloe vera extract and aloe vera leaf extract), apricot extract, apricot core extract, ginkgo extract, fennel extract, turmeric extract, oolong tea extract, rose fruit extract, echinacea leaf extract, scutellaria root extract, phellodendron bark extract, goldthread extract, barley extract, hypericum extract, white nettle extract, watercress extract, orange extract, Prunus amygdalus dulcis (sweet almond) oil, seaweed extract, carrot extract, Artemisia capillaris extract, glycyrrhiza extract, sabdariffa extract, Pyracantha fortuneana fruit extract, cinchona extract, cucumber extract, gardenia extract, Sasa albo-marginata extract, sophora root extract, walnut extract, grapefruit extract, Simmondsia chinensis (jojoba) seed oil, clematis extract, chlorella extract, mulberry bark extract, gentian extract, black tea extract, yeast extract, burdock extract, fermented rice bran extract, rice germ oil, comfrey extract, cowberry extract, asiasarum root extract, Bupleurum falcatum root extract, salvia extract, saponaria extract, bamboo grass extract, crataegus extract, zanthoxylum fruit extract, shiitake mushroom extract, rehmannia root extract, lithospermum root extract, perilla extract, linden extract, filipendula extract, peony root extract, calamus rhizome extract, birch extract, horsetail extract, ivy extract, hawthorn extract, Sambucus nigra extract, yarrow extract, peppermint extract, sage extract, mallow extract, cnidium rhizome extract, swertia herb extract, soy extract, jujube extract, wild thyme extract, green tea extract, clove extract, cogon extract, Citrus unshiu peel extract, angelica root extract, calendula extract, peach seed extract, bitter orange extract, houttuynia extract, tomato extract, natto extract, ginseng extract, garlic extract, wild rose extract, Hibiscus sabdariffa flower extract, ophiopogon tuber extract, parsley extract, witch hazel extract, pellitory extract, isodonis extract, matricaria extract, loquat extract, coltsfoot extract, butterbur scape extract, Poria cocos extract, butcher bloom extract, grape extract, Olea europaea (olive) fruit oil, propolis, luffa extract, safflower extract, peppermint extract, linden extract, peony extract, hop extract, pine extract, Zingiber officinale (ginger) root extract, horse chestnut extract, skunk cabbage extract, sapindaceae extract, balm mint extract, peach extract, cornflower extract, eucalyptus extract, saxifrage extract, coix seed extract, mugwort extract, lavender extract, apple extract, lettuce extract, lemon extract, Chinese milk vetch extract, rose extract and rosemary extract. In one embodiment, the composition comprises one or more of: Prunus amygdalus dulcis (sweet almond) oil, Simmondsia chinensis (jojoba) seed oil, Olea europaea (olive) fruit oil, Zingiber officinale (ginger) root extract, and combinations thereof. The additional botanical extract may be present in the compositions of the present disclosure in an amount ranging from between about 0.1 wt % and about 5 wt %, or between about 0.1 wt % and about 4 wt %, or between about 0.1 wt % and about 3 wt %, or between about 0.1 wt % and about 1 wt %.

In a particular embodiment, the composition of the present technology comprises from about 1 wt % to about 5 wt % CBD, or about 1 wt % CBD.

In some embodiments, the composition comprises a hemp-derived extract.

iii) Non Medicinal Cosmetic Ingredients

In some embodiments, the compositions of the present technology further comprise non-medicinal cosmetic ingredients. The non-medicinal cosmetic ingredients should be acceptable/suitable in the sense of being compatible with any other components in the composition and should not be deleterious to the subject. In some instances, the non-medicinal cosmetic ingredients may be incorporated into the compositions defined herein with the purpose of, for example, improving administration to the subject, improving bioavailability of the components of the composition, adjusting pH of the composition, modifying the texture of the composition, improving the stability of the composition, preventing microbial contamination of the composition, and the like. Examples of non-medicinal ingredients useful in the compositions of the present technology include, but are not limited to: absorbents, abrasives, anticaking agents, anticlumping agents, antifoaming agents, antimicrobial agents, binders, biological additives, buffering agents, bulking agents, chemical additives, cosmetic biocides, denaturants, cosmetic astringents, drug astringents, external analgesics, film formers, humectants, opacifying agents, fragrances, perfumes, pigments, colorings, essential oils, skin sensates, emollients, skin soothing agents, skin healing agents, pH adjusters, plasticizers, preservatives, preservative enhancers, propellants, reducing agents, skin-conditioning agents, skin penetration enhancing agents, skin protectants, solvents, suspending agents, emulsifiers, thickening agents, solubilizing agents, waxes, sunscreens, sunblocks, ultraviolet light absorbers or scattering agents, sunless tanning agents, antioxidants and/or radical scavengers, chelating agents, and sequestrants. Such other materials are known in the art.

The compositions of the present technology may comprise an emollient. For example, the emollient may be selected from one or more of the following classes: Triglycerides (e.g., Medium-chain triglycerides (MCT)), Triglyceride esters which include, but are not limited to, vegetable and animal fats and oils such as castor oil, cocoa butter, safflower oil, cottonseed oil, corn oil, olive oil, cod liver oil, almond oil, avocado oil, palm oil, sesame oil, squalene, kikui oil and soybean oil; Acetoglyceride esters, such as acetylated monoglycerides; Ethoxylated glycerides, such as ethoxylated glyceryl monostearate; Alkyl esters of fatty acids having 10 to 20 carbon atoms which include, but are not limited to, methyl, isopropyl, and butyl esters of fatty acids such as hexyl laurate, isohexyl laurate, isohexyl palmitate, isopropyl palmitate, methyl palmitate, decyloleate, isodecyl oleate, hexadecyl stearate decyl stearate, isopropyl isostearate, methyl isostearate, diisopropyl adipate, diisohexyl adipate, dihexyldecyl adipate, diisopropyl sebacate, lauryl lactate, myristyl lactate, and cetyl lactate; Alkenyl esters of fatty acids having 10 to 20 carbon atoms such as oleyl myristate, oleyl stearate, and oleyl oleate; Fatty acids having 10 to 20 carbon atoms such as pelargonic, lauric, myristic, palmitic, stearic, isostearic, hydroxystearic, oleic, linoleic, ricinoleic, arachidic, behenic, and erucic acids; Fatty alcohols having 10 to 20 carbon atoms such as lauryl, myristyl, cetyl, hexadecyl, stearyl, isostearyl, hydroxystearyl, oleyl, ricinoleyl, behenyl, erucyl, and 2-octyl dodecanyl alcohols; Lanolin and lanolin derivatives such as lanolin, lanolin oil, lanolin wax, lanolin alcohols, lanolin fatty acids, isopropyl lanolate, ethoxylated cholesterol, propoxylated lanolin alcohols, acetylated lanolin alcohols, lanolin alcohols linoleate, lanolin alcohols ricinoleate, acetate of lanolin alcohols ricinoleate, acetate of ethoxylated alcohols-esters, hydrogenolysis of lanolin, ethoxylated hydrogenated lanolin, and liquid and semisolid lanolin absorption bases; Polyhydric alcohol esters such as ethylene glycol mono and di-fatty acid esters, diethylene glycol mono- and di-fatty acid esters, polyethylene glycol (200-6000) mono- and di-fatty acid esters, propylene glycol mono- and di-fatty acid esters, polypropylene glycol 2000 monooleate, polypropylene glycol 2000 monostearate, ethoxylated propylene glycol monostearate, glyceryl mono- and di-fatty acid esters, polyglycerol polyfatty esters, ethoxylated glyceryl monostearate, 1,2-butylene glycol monostearate, 1,2-butylene glycol distearate, sorbitan fatty acid esters, and polyoxyethylene sorbitan fatty acid esters; Wax esters such as beeswax, spermaceti, myristyl myristate, stearyl stearate; Beeswax derivatives such as polyoxyethylene sorbitol beeswax which are reaction products of beeswax with ethoxylated sorbitol of varying ethylene oxide content, forming a mixture of ether esters; Vegetable waxes including, but not limited to, carnauba and candelilla waxes; Phospholipids such as lecithin and derivatives; Sterols including, but not limited to, cholesterol and cholesterol fatty acid esters; and Amides such as fatty acid amides, ethoxylated fatty acid amides, and solid fatty acid alkanolamides.

The compositions of the present technology may comprise a surfactant. The surfactant may be synthesized or isolated from a natural source. For example, the surfacatant may be selected from one or more of the following classes: anionic surfactants, nonionic surfactants, cationic surfactants, amphoteric surfactants, plant-derived surfactants, coconut-derived surfactants, palm-derived surfactants, vegetable derived surfactants, coco glucoside, decyl glucoside, hexyl glucoside, capryl glucoside, lauryl glucoside, disodium laureth sulfosuccinate, coco betaine, sodium coco sulfate, Plantapon® SF, soap nuts or berries, aritha, yucca extract, shikakai powder, saponins, and soapwort. In some such embodiments, the compositions of the present technology comprise a saponin. In some such embodiments, the compositions of the present technology comprise a natural food-grade surfactant such as Quillaja saponin (Q-Naturale®). In some such embodiments, the compositions of the present technology comprise a synthetic surfactant such as Tween 80.

The compositions of the present technology may comprise a carrier oil. The carrier oil is, optionally, food grade, does not adversely affect product quality (such as appearance, taste, texture, or stability), protects from chemical degradation during storage and distribution, and/or increases bioavailability following application by, for example, aiding skin penetration of a cream. Carrier oils can help stabilize emulsions.

In embodiments of the compositions of the present technology, the carrier oil may be synthesized or isolated from a natural source. In some embodiments, the carrier oil is a natural oil as known in the art, such as an edible vegetable oil. In some alternative embodiments, the carrier oil is a synthetic edible oil, such as a hydrogenated vegetable oil, a medium-chain triglyceride (MCT) oil, and the like. For example, the carrier oil may be selected from one or more of the following classes, without limitation: medium-chain triglycerides (MCT) oil, medium-chain fatty acids (e.g., caproic acid, caprylic acid, capric acid, lauric acid, long-chain triglycerides (LCT oil), long chain fatty acids (e.g., myristic acid, palmitic acid, stearic acid, arachidic acid, linoleic acid), glycerine/glycerol, Maisine®CC, glycerol monolinoleate, coconut oil, corn oil, canola oil, olive oil, avocado oil, vegetable oil, flaxseed oil, palm oil, palm kernel oil, peanut oil, sunflower oil, rice bran oil, safflower oil, jojoba oil, argan oil, grapeseed oil, castor oil, wheat germ oil, arnica oil, peppermint oil, hemp oil, sesame oil, pomegranate seed oil, terpenes, terpenoids, beta-myrcene, linalool, α-pinene, beta-pinene, beta-caryophyllene, caryophyllene oxide, α-humulene, nerolidol, D-limonene, L-limonene, para-cymene, eugenol, farnesol, geraniol, phytol, menthol, terpineol, α-terpineol, benzaldehyde, hexyl acetate, methyl salicylate, eucalyptol, ocimene, terpinolene, α-terpinene, isopulegol, guaiol, α-bisabolol and combinations thereof. Other suitable carrier oils include Labrasol, LabrafacLipophile WL 1349, Labrail M1944, Peceol, Plurol Oliqiue CC 497, Transcutol HP, Tween 80, Gelucire 48/16, and combinations thereof. Carrier oils may also be in the form of an oil powder, such as a plant or animal-derived oil powder (such as rice bran oil powder, coconut oil powder, grape seed oil powder, cranberry seed oil powder, chia seed oil powder, flaxseed oil powder, MCT oil powder, hydrolyzed collagen powder, saw palmetto oil powder, safflower oil powder, evening primrose seed oil powder, fish oil powder, or the like). In some embodiments, the carrier oil is medium-chain triglycerides (MCT), e.g., MCT oil or MCT oil powder.

In some embodiments of compositions of the present technology, the weight ratio of the cannabinoid to carrier oil is about 5:1 or about 1:5. In alternative embodiments, the weight ratio of cannabinoid to carrier oil is about 1:1.

The compositions of the present technology may comprise an emulsifier. The emulsifier may be synthesized or isolated from a natural source. Emulsifiers may be provided in the liquid, granule or powder form. In some embodiments, the emulsifier is provided in the form of an emulsifying powder. A wide range of emulsifiers and emulsifying powders suitable for spray granulation, milling, liquefying, pressing or spray drying may be used. For example, the emulsifying powder may be selected from one or more of the following classes: lecithin, acconon mixtures, capmul MCG, propylene glycol esters, caprol polyglycerol esters, glycerides, glycerol, captex medium chain esters, Kolliphor, castor oil, polysorbate 80, hydroxypropylcellulose (HPC), and the like.

In some embodiments, the compositions of the present technology are formulated for topical administration. For example, the composition may be in the form of a patch, a matrix, a wax, an ointment, a cream, a serum, a balm, a suspension, a lotion, a paste, a gel, a spray, an aerosol, a foam, or an oil. In some such embodiments, the composition includes one or more formulating agents suitable for administration in topical form such as, without limitation, binders, fillers, bulking agents, excipients, pressing agents, press release agents, powders, dispersing agents, disintegrants, lubricants, glidants, surfactants, emulsifiers, solubilizing agents, and fillers.

In some embodiments, compositions described herein are suitable for transdermal administration. In another embodiment, transdermally administrable compositions are adapted for administration in and/or around the abdomen, back, chest, legs, arms, scalp, face or other suitable skin surface and may include formulations of the compositions disclosed herein administered in patches, ointments, creams, suspensions, lotions, pastes, gels, sprays, aerosols, foams, serums, balms, or oils.

Compositions of the disclosure containing can be prepared by any technique known to a person of ordinary skill in the art of pharmacy, pharmaceutics, cosmetics, drug delivery, pharmacokinetics, medicine or other related discipline that comprises admixing non-medicinal ingredients with a drug or therapeutic agent, a cosmetic ingredient, and the like.

In certain embodiments, the compositions of the present technology are suitable for administration by inhalation and/or nasal administration. In an embodiment, compositions are formulated into a spray, e.g., into a form which is easily sprayable. In an embodiment, compositions are dispensed in the form of an aerosol spray.

In certain embodiments, the compositions of the present technology, or at least a portion thereof, are encapsulated. This delivery mechanism allows the composition to be released consistently over a prolonged time, for example throughout the night, as the encapsulated composition remains enclosed until the encapsulate is broken. Breaking the encapsulate allows additional composition to be released. Many different encapsulation technologies are available and may be used in the compositions of the present technology. For examples, encapsulates may be synthetic in origin (e.g., polymer shells), may contain animal derivatives (e.g., lecithin), or may contain only plant derivatives (e.g., vegan encapsulates). Encapsulates may be “broken” to release composition or fragrance through heat, moisture, or friction. In one embodiment, the encapsulate is between about 5 to about 25 microns in size. For example, the encapsulate may be about 5 about 10, about 15, about 20, or about 25 microns in size.

In one embodiment, the composition of the present technology, or at least a portion thereof, is encapsulated such that the encapsulate is broken by friction, e.g., by the subject moving about during the night, allowing additional composition or fragrance to be released. In some such embodiments, the encapsulate comprises only plant-derived materials, e.g., is a vegan encapsulate. In one embodiment, the main components of the encapsulate wall include one or more of: Sodium Alginate (CAS 9005-38-3); Calcium Chloride (CAS 10043-52-4, EINECS 233-140-8); Riboflavin 5-Phosphate (CAS 146-17-8, EINECS 205-664-7); Genipin (CAS 6902-77-8); and combinations thereof. In one embodiment, the components of the encapsulate are 100% naturally derived.

In some embodiments, the compositions of the present technology are formulated into a matrix. In certain embodiments, the matrix may be a lipid matrix. Suitable lipid matrices include, without limitation, natural and/or synthetic oils, fatty acids and their derivatives, glycerides, fatty acid esters, glycolized fatty acid esters, fatty alcohols, sterols, waxes, hard fat, and/or combination thereof. Suitable natural oils include, without limitation, vegetable oil such as sunflower oil, olive oil, groundnut oil, and palm oil, as well as hydrogenated vegetable oils, including hydrogenated cottonseed oil. Suitable synthetic oils include, without limit, hydrophobic silicone, cyclomethicones, petroleum waxes or jellies, linear alkanes, lipophilic organic fluorinated oils, perhydrosqualene and/or mixtures thereof. Suitable fatty acids include, without limitation, stearic acid, benzoic acid, citric acid, iumaric acid, lactic acid, and maleic acid. Exemplary glycerides include, without limitation, monoglycerides, diglycerides, triglycerides, and combinations thereof, etc. with saturated or unsaturated chains having carbon numbers from C₆ to C₄₀, e.g. C₁₈ to C₂₄, C₈ to C₃₂, C₁₀ to C₂₄, C₁₀ to C₁₈, C₁₂ to C₁₈, etc.), hemisynthetic glycerides or glyceride derivatives with saturated or unsaturated medium to long chain lengths. Suitable waxes include, without limitation, Carnauba wax, Candelilla wax, Alfa wax, vegetable waxes, rice wax, hydrogenated jojoba wax or floral absolute waxes, beeswaxes and modified beeswaxes, microcrystalline wax, and paraffin wax. Suitable fatty alcohols include fatty alcohols with high molecular weight (e.g. cetanol, myristoyl alcohol, stearyl alcohol). Esters of acids and alcohols with high molecular weight include, without limitation, esters of linear and saturated acids with even carbon numbers from C₁₄ to C₂₀, and linear and saturated alcohols with even carbon numbers from C₁₄ to C₃₂. Suitable lipid matrix materials may also include clays or their oily dispersions, gums of phenylated silicones, starches, and/or fat structuring agents for the purpose of adjusting consistency. The lipid matrix may also include a certain number of compounds such as mineral fillers, to modulate density and plasticity. The mineral fillers may be, for example, talc and/or kaolin. The matrix material may include mixtures of materials, such as mixtures of any of the foregoing.

Compositions of the disclosure containing can be prepared by any technique known to a person of ordinary skill in the art of pharmacy, pharmaceutics, cosmetics, drug delivery, pharmacokinetics, medicine or other related discipline that comprises admixing non-medicinal ingredients with a drug or therapeutic agent, a functional agent, a cosmetic ingredient, and the like.

In some embodiments, compositions of the present technology comprise a penetration enhancing agent for transdermal or topical delivery. Suitable penetration enhancing agents include, without limitation, C₈-C₂₂ fatty acids such as isostearic acid, octanoic acid, and oleic acid; C₈-C₂₂ fatty alcohols such as oleyl alcohol and lauryl alcohol; lower alkyl esters of C₈-C₂₂ fatty acids such as ethyl oleate, isopropyl myristate, butyl stearate, and methyl laurate; di(lower)alkyl esters of C₆-C₂₂ diacids such as diisopropyl adipate; monoglycerides of C₈-C₂₂ fatty acids such as glyceryl monolaurate; tetrahydrofurfuryl alcohol polyethylene glycol ether; polyethylene glycol, propylene glycol; 2-(2-ethoxyethoxy)ethanol; diethylene glycol monomethyl ether; alkylaryl ethers of polyethylene oxide; polyethylene oxide monomethyl ethers; polyethylene oxide dimethyl ethers; dimethyl sulfoxide; glycerol; ethyl acetate; acetoacetic ester; N-alkylpyrrolidone; and terpenes.

In one embodiment, the compositions described herein comprise an antimicrobial preservative. Suitable antimicrobial preservatives include, without limitation, acids, benzoic acid, phenolic acid, sorbic acids, alcohols, benzethonium chloride, bronopol, butylparaben, cetrimide, chlorhexidine, chlorobutanol, chlorocresol, cresol, ethylparaben, imidurea, methylparaben, phenol, phenoxyethanol, phenylethyl alcohol, phenylmercuric acetate, phenylmercuric borate, phenylmercuric nitrate, potassium sorbate, propylparaben, sodium propionate, or thimerosal. The antimicrobial preservative, if present, is present in an amount of from about 0.1% to about 5%, from about 0.2% to about 3%, or from about 0.3% to about 2%, by weight. In some embodiments, the anti-microbial preservative, if present, is present in an amount of about 0.1%, about 0.2%, about 0.4%, about 0.6%, about 0.8%, about 1%, about 1.2%, about 1.4%, about 1.6%, about 1.8%, about 2%, about 2.2%, about 2.4%, about 2.6%, about 2.8%, about 3.0%, about 3.2%, about 3.4%, about 3.6%, about 3.8%, about 4%, about 4.2%, about 4.4%, about 4.6%, about 4.8%, or about 5%.

In some embodiments, the compositions described herein comprise one or more antioxidant preservative. For example, an antioxidant may be used to preserve integrity and/or appearance of the composition. In some embodiments, the one or more antioxidant serves to stabilize the composition. In some embodiments, the one or more antioxidant serves to prevent discoloration of the composition.

Compositions described herein may also optionally include one or more taste enhancers, such as sweeteners, including aspartame, acesulfame potassium, sucralose and saccharin or taste masking agents, such as flavorings. Depending on the desired result, a person of ordinary skill in the art of pharmacy, pharmaceutics, drug delivery, pharmacokinetics, medicine or other related discipline that comprises admixing an excipient with a drug or therapeutic agent to a composition would be able to select the appropriate taste enhancer or taste making agent and the relative concentration of the taste enhancer or taste masking agent.

In some embodiments, the non-medicinal cosmetic ingredient useful in the compositions of the present technology is Phenoxyethanol. Phenoxyethanol is an oily, slightly sticky liquid with a faint rose-like scent. It is used as a preservative in a wide variety of both leave-on and rinse-off cosmetics and personal care products, including skin care, eye makeup, fragrances, blushers, foundations, lipstick, bath soaps, and detergents, among others. It is highly effective in preventing the growth of fungi, bacteria, and yeast that could cause products to spoil, and thus enhances products' shelf life and safety. Phenoxyethanol is often synthesized for commercial use but it can also be found naturally in materials such as green tea. In some such embodiments, the composition of the present technology thus comprises CBD or a derivative thereof, at least one active cosmetic ingredient; and Phenoxyethanol or a derivative thereof.

In some embodiments, the non-medicinal cosmetic ingredient useful in the compositions of the present technology is Sodium Metabisulfite. Sodium Metabisulfite is an inorganic sulfite salt with antioxidant properties used in a variety of personal care products such as skin care products, often as a preservative. In some such embodiments, the composition of the present technology thus comprises CBD or a derivative thereof; at least one active cosmetic ingredient; and Sodium Metabisulfite.

In some embodiments, the non-medicinal cosmetic ingredient useful in the compositions of the present technology is dimethyl isosorbide. Dimethyl Isosorbide (DMI) is a high purity solvent and carrier which offers a safe, effective delivery enhancement mechanism for active ingredients such as antioxidants in cosmetics and personal care products. It may be plant-based or synthetic. It is also used to decrease the thickness of products, such as keeping a liquid in its fluid form rather than allowing it to revert to a lotion (a thicker emulsion). In some such embodiments, the composition of the present technology thus comprises CBD or a derivative thereof; at least one active cosmetic ingredient; and dimethyl isosorbide.

In some embodiments, the non-medicinal cosmetic ingredient useful in the compositions of the present technology is Tartaric acid. Tartaric Acid is an organic carboxylic acid. It occurs naturally in many fruits, particularly grapes and tamarinds. Salts of Tartaric Acid are called Tartrates. These salts include Calcium Tartrate, Potassium Tartrate, Potassium Sodium Tartrate and Disodium Tartrate. Tartaric Acid and its salts are used as pH adjusters in cosmetics and personal care products such as skin care products. In some such embodiments, the composition of the present technology thus comprises CBD or a derivative thereof; at least one active cosmetic ingredient; and tartaric acid.

In some embodiments, the non-medicinal cosmetic ingredient useful in the compositions of the present technology is Magnesium chloride. Magnesium Chloride is a colorless crystalline material that occurs naturally as the mineral bischofite. In cosmetics and personal care products, Magnesium Chloride is used as a flavoring agent and a viscosity increasing agent. Magnesium Chloride may be present in the compositions of the present disclosure in an amount ranging from between about 0.1 wt % and about 5 wt %, or between about 0.1 wt % and about 3 wt %, or between about 0.5 wt % and about 2 wt %, or between about 0.5 wt % and about 1 wt %. In some such embodiments, the composition of the present technology thus comprises CBD or a derivative thereof; at least one active cosmetic ingredient; and magnesium chloride.

In some embodiments, the non-medicinal cosmetic ingredient useful in the compositions of the present technology is Benzyl alcohol. Benzyl Alcohol is an organic alcohol found in many fruits, teas and essential oils. Benzyl Alcohol has a hydroxyl group (—OH), while the related compound, Benzoic Acid has a carboxyl group (—COOH). Sodium Benzoate, Calcium Benzoate and Potassium Benzoate are salts of Benzoic Acid. Benzyl Benzoate is an ester of Benzyl Alcohol and Benzoic Acid. Benzyl Alcohol, Benzoic Acid and its salts, and Benzyl Benzoate are used in a wide variety of cosmetics and personal care products such as makeup and skin care products as a fragrance, a preservative, and/or a solvent. In some such embodiments, the composition of the present technology thus comprises CBD or a derivative thereof; at least one active cosmetic ingredient; and benzyl alcohol.

In some embodiments, the non-medicinal cosmetic ingredient useful in the compositions of the present technology is Butylene glycol. Butylene Glycol, Hexylene Glycol, Ethoxydiglycol and Dipropylene Glycol are glycols or glycol ethers used as solvents and viscosity decreasing agents in cosmetics and personal care products. In some such embodiments, the composition of the present technology thus comprises CBD or a derivative thereof; at least one active cosmetic ingredient; and Butylene glycol.

In some embodiments, the non-medicinal cosmetic ingredient useful in the compositions of the present technology is Xanthan Gum. Xanthan Gum is a polysaccharide derived from glucose or corn syrup. It is used in a wide variety of cosmetics and personal care products including makeup and skin care products as an emulsifying agent, emulsion stabilizer, viscosity increasing agent, and/or binder. In some such embodiments, the composition of the present technology thus comprises CBD or a derivative thereof; at least one active cosmetic ingredient; and Xanthan Gum.

In some embodiments, the non-medicinal cosmetic ingredient useful in the compositions of the present technology is Polysorbate 20. Polysorbate 20 and other related Polysorbate ingredients (Polysorbate 21, Polysorbate 40, Polysorbate 60, Polysorbate 61, Polysorbate 65, Polysorbate 65, Polysorbate 80, Polysorbate 81, Polysorbate 85) are hydrophilic, nonionic surfactants. They are used in a wide variety of cosmetics and personal care products. In some such embodiments, the composition of the present technology thus comprises CBD or a derivative thereof; at least one active cosmetic ingredient; and Polysorbate 20.

In some embodiments, the non-medicinal cosmetic ingredient useful in the compositions of the present technology is Disodium EDTA. EDTA (ethylenediamine tetraacetic acid) and its salts, Calcium Disodium EDTA, Diammonium EDTA, Dipotassium EDTA, Disodium EDTA, TEA-EDTA, Tetrasodium EDTA, Tripotassium EDTA and Trisodium EDTA, and the related ingredients HEDTA (hydroxyethyl ethylenediamine triacetic acid) and its trisodium salt, Trisodium HEDTA, are metal chelators widely used in cosmetics and personal care products, primarily as preservatives and as foaming agents. In some such embodiments, the composition of the present technology thus comprises CBD or a derivative thereof; at least one active cosmetic ingredient; and Disodium EDTA.

In some embodiments, the non-medicinal cosmetic ingredient useful in the compositions of the present technology is Sodium Hydroxide. Sodium hydroxide (and other bases such as calcium hydroxide, magnesium hydroxide and potassium hydroxide) is used widely in cosmetics and personal care products to control the pH. In some such embodiments, the composition of the present technology thus comprises CBD or a derivative thereof; at least one active cosmetic ingredient; and Sodium hydroxide.

In some embodiments, the non-medicinal cosmetic ingredient useful in the compositions of the present technology is Dehydroacetic Acid. Dehydroacetic Acid and Sodium Dehydroacetate are used in a wide range of cosmetics and personal care products for their antimicrobial properties. In some such embodiments, the composition of the present technology thus comprises CBD or a derivative thereof; at least one active cosmetic ingredient; and Dehydroacetic Acid.

In some embodiments, the non-medicinal cosmetic ingredient useful in the compositions of the present technology is Stearic acid. Stearic acid is a naturally occurring fatty acid used as a surfactant and an emulsifying agent. In some such embodiments, the composition of the present technology thus comprises CBD or a derivative thereof; at least one active cosmetic ingredient; and stearic acid.

In some embodiments, the non-medicinal cosmetic ingredient useful in the compositions of the present technology is Cetyl Alcohol. Cetyl Alcohol is a naturally occurring fatty alcohol used as an emulsifier and a viscosity increasing agent to stabilize foams. In some such embodiments, the composition of the present technology thus comprises CBD or a derivative thereof; at least one active cosmetic ingredient; and Cetyl alcohol.

In some embodiments, the non-medicinal cosmetic ingredient useful in the compositions of the present technology is Octyldodecanol. Octyldodecanol is a long chain fatty alcohol commonly used as an emulsifier. It can also act as a lubricant on the skin's surface, giving the skin a soft, smooth appearance. In some such embodiments, the composition of the present technology thus comprises CBD or a derivative thereof; at least one active cosmetic ingredient; and Octyldodecanol.

In some embodiments, the non-medicinal cosmetic ingredient useful in the compositions of the present technology is Ceteth-20. Ceteth-20 is prepared by reacting 20 moles of ethylene oxide with cetyl alcohol. It is used as a solubilizing agent and an emulsifier. In some such embodiments, the composition of the present technology thus comprises CBD or a derivative thereof; at least one active cosmetic ingredient; and Ceteth-20.

In some embodiments, the non-medicinal cosmetic ingredient useful in the compositions of the present technology is Steareth-20. Steareth-20 is a polyethylene glycol ether of stearic acid and is prepared by reacting 20 moles of ethylene oxide with stearyl alcohol. It is used as an emulsifying agent. In some such embodiments, the composition of the present technology thus comprises CBD or a derivative thereof; at least one active cosmetic ingredient; and Steareth-20.

In some embodiments, the non-medicinal cosmetic ingredient useful in the compositions of the present technology is PEG-75 Stearate. PEG-75 Stearate (also referred to as PEG-75 Propylene Glycol Stearate) is a polyethylene glycol (PEG) ether of propylene glycol stearate. It can be used as a solubilizing agent, a moisturizer, and/or an emollient. In some such embodiments, the composition of the present technology thus comprises CBD or a derivative thereof; at least one active cosmetic ingredient; and PEG-75 Stearate.

In some embodiments, the non-medicinal cosmetic ingredient useful in the compositions of the present technology is Acrylates/C10-30 Alkyl Acrylate Crosspolymer is a synthetic ingredient used as a thickening agent, texture enhancer, film-forming agent, and emulsifier in cosmetics and personal care products, such as moisturizers, cleansers, and sunscreen. In some such embodiments, the composition of the present technology thus comprises CBD or a derivative thereof; at least one active cosmetic ingredient; and C10-30 Alkyl Acrylate Crosspolymer.

In some embodiments, the non-medicinal ingredients used in the compositions of the present technology are naturally occurring or are naturally derived or are a combination thereof. As used herein, the expression “naturally occurring” refers to a natural product that is delivered in a natural form. The expression “naturally derived”, as used herein, refers to some ingredients derived from nature that have been used to artificially create a product that is delivered in an unnatural form.

It should be understand that some active cosmetic ingredients can be used as non-medicinal cosmetic ingredients in certain embodiments, and vice-versa, some non-medicinal cosmetic ingredients can be used as active cosmetic ingredients in certain embodiments. For example, due to its antioxidant properties, tocopherol may be used as a non-medicinal cosmetic ingredient to stabilize a composition, e.g., by preventing discoloration, and as an active ingredient to provide desired antioxidant protection to the skin. Other such examples are known and the person skilled in the art of formulating cosmetic and personal care products will select ingredients of the compositions to provide the desired properties accordingly.

iv) Formulation and Methods of Use

It is known that cannabinoids, including cannabidiol, have anxiolytic, antidepressant, anti-inflammatory, calming, relaxing, and anti-insomnia effects, which may be effective in promoting health or well-being of a subject, as well as effects useful for the treatment of acne. Active cosmetic ingredients disclosed herein are also known to enhance health or well-being of a subject and/or to make a subject look, smell or feel better. As such, in some embodiments of the present technology, the compositions of the present technology are used to promote health, well-being and/or appearance.

The compositions of the present technology may be provided in a wide variety of cosmetics and personal care products such as, without limitation, skin care products (e.g., body and hand creams and lotions, cleansers, face and neck creams and lotions, moisturizers, foot powders and sprays, masks), sunscreens, sunless tanners, shaving creams, aftershave lotions, soaps, deodorants, shampoos, perfumes, powders, and makeups. It should be understood that cosmetic compositions of the present technology may be in the form of any such personal care product as is appropriate for its intended use and effect.

In some embodiments of the present technology, there is provided a method of enhancing health, well-being, or appearance of a subject, comprising administering to said subject an effective amount of the composition(s) provided herein. Compositions may be administered as creams, ointments, serums, lotions, or oils. For example, compositions may be administered as a skin care product providing one or more of the following: reducing uneven skin tone; reducing signs of aging; revitalizing skin; smoothing and/or brightening skin; neutralizing free radicals; reducing wrinkles and fine lines; detoxifying skin; improving elasticity; improving texture, tone, and/or color of skin; treating or reducing acne; unclogging blocked pores; stimulating collagen production; moisturizing or hydrating skin; exfoliating skin; and generally improving appearance of the skin. Alternatively, compositions may be administered as a massage oil for muscle therapy. As another example, compositions may be administered topically to promote relaxation or sleep.

In some embodiments of the present technology, there is provided a method of improving appearance of the skin of a subject, comprising administering to said subject an effective amount of the composition(s) provided herein. In some embodiments, the method smooths or brightens the skin, and/or increases skin radiance, in the subject. In some embodiments, the method improves pigmentation (e.g., reduces redness) or skin tone and/or reduces redness of the subject's skin. In some embodiments, the method exfoliates the skin. In some embodiments, the method improves the clarity, texture, color, and/or tone of the skin. In some embodiments, the method stimulates collagen production.

In some embodiments of the present technology, there is provided a method of moisturizing or hydrating the skin of a subject, comprising administering to said subject an effective amount of the composition(s) provided herein.

In some embodiments of the present technology, there is provided a method of reducing the signs of aging in a subject, comprising administering to said subject an effective amount of the composition(s) provided herein. In some embodiments, the method reduces the appearance of fine lines and/or wrinkles.

In some embodiments of the present technology, there is provided a method of treating an inflammatory skin condition in a subject, comprising administering to said subject an effective amount of the composition(s) provided herein. Inflammatory skin conditions that may be treated using compositions of the present technology include, without limitation: cold sores; acne; psoriasis, and eczema. Accordingly, in some embodiments of the present technology, there is provided a method of treating acne in a subject, comprising administering to said subject an effective amount of the composition(s) provided herein. In some embodiments, the method unclogs blocked pores in a subject. In other embodiments, the method reduces the production of sebum in a subject. In other embodiments of the present technology, there is provided a method of treating eczema in a subject, comprising administering to said subject an effective amount of the composition(s) provided herein. In other embodiments of the present technology, there is provided a method of treating psoriasis in a subject, comprising administering to said subject an effective amount of the composition(s) provided herein.

In some instances, the compositions of the present technology are used in methods for treating anxiety, insomnia, depression, and related disorders. In some implementations of these embodiments, the methods of the present technology comprise administering the composition of the present disclosure to a subject suffering from anxiety, insomnia, depression, or a related disorder.

Suitable dosages may be readily determined by one skilled in the art such as, for example, a physician, a veterinarian, a scientist, and other medical and research professionals. For example, one skilled in the art can begin with a low dosage that can be increased until reaching the desired treatment outcome or result. Alternatively, one skilled in the art can begin with a high dosage that can be decreased until reaching a minimum dosage needed to achieve the desired treatment outcome or result.

Suitable amounts of the cannabidiol (CBD), derivative, intermediate, or prodrug thereof, and combinations thereof used in the dosage forms of the present disclosure will depend upon many factors including, for example, age and weight of an individual, specific cannabidiol (CBD), derivative, intermediate, or prodrug thereof, and combinations thereof to be used, nature of a composition, intended means of delivery, etc. Ultimately, a suitable amount can be readily determined by one skilled in the art. For example, one skilled in the art can begin with a low amount that can be increased until reaching the desired result or effect. Alternatively, one skilled in the art can begin with a high dosage that can be decreased until reaching a minimum dosage needed to achieve the desired result or effect.

In some instances, the administration comprises topical or transdermal administration, e.g., administration of a form such as a lotion, cream, ointment, gel, paste, liniment or balm, serum, drops, skin patch, aerosol, spray, and the like.

In one embodiment, compositions described herein are suitable for transdermal administration. In another embodiment, transdermally administrable compositions are adapted for administration in and/or around the abdomen, back, chest, legs, arms, scalp or other suitable skin surface and may include formulations of the compositions disclosed herein is administered in patches, ointments, creams, suspensions, lotions, pastes, gels, sprays, aerosols, foams, or oils.

In one embodiment, compositions described herein are suitable for topical administration. In another embodiment, topical administrable compositions are adapted for administration in and/or around the face, abdomen, back, chest, legs, arms, scalp, or other suitable skin surface and may include formulations of the compositions disclosed herein is administered in patches, ointments, creams, suspensions, lotions, pastes, gels, sprays, foams, or oils.

In one embodiment, compositions described herein are suitable for ocular administration. In another embodiment, compositions described herein that are ocularly administrable may include formulations of the compositions disclosed herein is placed in ointments, suspensions, solutions, gels, or sprays.

In one embodiment, compositions described herein are suitable for nasal administration. In another embodiment, compositions described herein that are nasally administrable may include formulations of the compositions disclosed herein is placed in ointments, suspensions, solutions, lotions, pastes, gels, sprays, or mists.

In one embodiment, compositions described herein are suitable for inhalation administration. In another embodiment, compositions described herein that are inhaled administrable may include formations of the compositions disclosed herein is placed in an inhaler, vaporizer, vape pen, aerosol spray, or the like.

As used herein, the term “treating” or “treatment” of any disease, disorder or condition includes, in some embodiments, arresting or reducing the development of the disease, disorder or condition, and/or ameliorating at least one symptom thereof. In certain embodiments “treating” or “treatment” refers to ameliorating at least one physical parameter, which may or may not be discernible by the patient. In certain embodiments, “treating” or “treatment” refers to inhibiting the disease or disorder, either physically, (e.g., stabilization of a discernible symptom), physiologically, (e.g., stabilization of a physical parameter), or both. In certain embodiments, “treating” or “treatment” refers to delaying the onset of the disease or disorder. In certain embodiments, “treating” or “treatment” refers to slowing or halting the progression of the disease or disorder. The term “treating” thus refers to any indicia of success in the treatment or amelioration of an injury, pathology or condition, including any objective or subjective parameter such as for example: abatement; remission; diminishing of symptoms or making the injury, pathology or condition more tolerable to the subject; slowing in the rate of degeneration or decline; making the final point of degeneration less debilitating; improving a subject's physical or mental well-being; slowing or halting disease progression; or, in some situations, healing or curing a disease. The treatment or amelioration of symptoms can be based on objective or subjective parameters, including the results of a physical examination, a patient's report, or any suitable test for the disease or disorder being treated.

In some embodiments, the compositions of the present technology are used in methods for the treatment of muscle pain. In some implementations of these embodiments, the methods of the present technology comprise administering the composition of the present disclosure to a subject in need thereof In some further implementations, the compositions of the present technology are used in methods for relieving tired and sore muscles and/or for providing relief from discomfort and muscle tension. In some instances, muscle relief is achieved while nourishing the skin.

In some embodiments, the compositions of the present technology are used in methods for skin care. In some implementations of these embodiments, the methods of the present technology comprise administering the composition of the present disclosure to a subject in need thereof. In some further implementations, the compositions of the present technology are used in methods for moisturizing and hydrating skin. In some instances, the compositions of the present technology are used in methods for achieving one or more of: reducing the appearance of wrinkles, smoothing skin, brightening skin, improving skin cell renewal, and increasing collagen production in skin.

In some instances, the administration comprises topical or transdermal administration, e.g., administration of a form such as a lotion, cream, ointment, gel, paste, liniment or balm, serum, drops, skin patch, spray, aerosol spray, and the like.

In some instances, the administration comprises nasal administration or administration by inhalation, e.g., administration of a form such as a spray, an aerosol, a pillow spray, and the like.

It should be understood that compositions of the present technology can be administered using any device or kit for delivery known in the art. In an embodiment, the compositions described herein are provided in a device for delivery to a subject in need thereof. The device may include any container suitable for holding a maximum amount of the composition, a provisioning mechanism for providing a dose of the composition to the subject, and a metering system for transporting the composition to the provisioning mechanism, such that the amount of composition delivered to the subject is controlled by the metering system. The device may deliver any amount of the composition held in the container. In a further embodiment, the amount delivered to the subject is less than the maximum amount held in the container. In some embodiments, the amount delivered to the subject is the same as the maximum amount held in the container. The delivery of the composition from the device to the subject may be controlled by the subject or by another.

A “subject” may be a human or an animal, such as without limitation a cat, a dog, a monkey, a mouse, a rat, or a rodent. In some embodiments, a subject is a patient suffering from insomnia or another sleep disorder, or otherwise in need of treatment for symptoms or sequelae of insomnia and associated conditions.

In some embodiments, the compositions of the present disclosure are formulated to a form suitable for administration to a subject (e.g., human, animal) In some implementations of these embodiments, the compositions as defined herein are formulated in a dosage form selected from a group consisting of liquid, solid, gas, oral, pill, tablet, capsule, buccal, sub-lingual, orally-disintegrating, thin film, liquid solution, suspension, powder or liquid or solid crystals, pastes, inhalational, aerosol, inhaler, nebulizer, smoking, vaporizer, parenteral, intradermal, intramuscular, intraosseous, intraperitoneal, intravenous, subcutaneous, topical, cream, gel, liniment or balm, lotion, ointment, serum, drops, skin patch, vaginal, suppository, pessary, rectal and any combination thereof. In some embodiments the compositions defined herein are formulated in any form suitable for inhalation. In some implementations of these embodiments, the compositions of the present disclosure are formulated in a form suitable for inhalation or nasal administration to a subject, such as a sprayable or aerosol form. In some implementations of these embodiments, the compositions of the present disclosure are formulated in a form suitable for topical or transdermal administration to a subject, such as a cream, lotion, serum or ointment. The most suitable route of administration in any given case will depend on the nature and severity of the condition being treated.

In some embodiments, the compositions of the present disclosure are formulated in a unit dosage form for ease of administration and uniformity of dosage. The term “unit dosage form” refers to a physically discrete unit suitable as unitary dosages for human subjects and other animals, each unit containing a predetermined quantity of active material (e.g., cannabinoid, active cosmetic ingredient) calculated to produce the desired therapeutic or cosmetic effect, in association with excipients and/or other components of the composition. The specification for the unit dosage forms of the technology may vary and are dictated by and directly dependent on (a) the unique characteristics of the active compound(s) and the particular therapeutic or cosmetic effect to be achieved, and (b) the limitations inherent in the art of formulating such active compound(s) (e.g., cannabinoid).

Suitable dosages of the compositions for use in the methods of the present disclosure will depend upon many factors including, for example, age and weight of an individual, at least one precise event requiring professional consultation, severity of an event, specific composition to be used, nature of a composition, route of administration and combinations thereof. Ultimately, a suitable dosage can be readily determined by one skilled in the art such as, for example, a physician, a veterinarian, a scientist, a cosmetologist, an esthetician, and other professionals. For example, one skilled in the art can begin with a low dosage that can be increased until reaching the desired treatment outcome or result. Alternatively, one skilled in the art can begin with a high dosage that can be decreased until reaching a minimum dosage needed to achieve the desired treatment or cosmetic outcome or result.

In a particular embodiment, the composition of the present technology comprises from about 1 to about 3 wt % CBD.

In another particular embodiment, the composition of the present technology comprises from about 3 to about 5 wt % CBD.

In a particular embodiment, the composition of the present technology comprises about 100 mg/mL CBD.

### EXAMPLES

The examples below are given so as to illustrate the practice of various embodiments of the present disclosure. They are not intended to limit or define the entire scope of this disclosure. It should be appreciated that the disclosure is not limited to the particular embodiments described and illustrated herein but includes all modifications and variations falling within the scope of the disclosure as defined in the appended embodiments.

### Example 1—Skin Care Compositions

Compositions formulated as creams, ointments, serums or lotions for use in skin care, particularly as anti-aging, anti-wrinkle, and/or moisturizing creams, are provided. Skin care compositions may have use for one or more of the following: reducing uneven skin tone; reducing signs of aging; revitalizing skin; smoothing and/or brightening skin; neutralizing free radicals; reducing wrinkles and fine lines; detoxifying skin; improving elasticity; improving texture, tone, and/or color of skin; treating or reducing acne; unclogging blocked pores; stimulating collagen production; moisturizing or hydration; exfoliating skin; and generally improving appearance of the skin.

Test Composition A:

In an embodiment, the composition may include between about 0.5 wt % and about 10 wt % CBD; ubiquinone (e.g., between about 0.1 wt % and about 1 wt %); caprylic/capric triglyceride (e.g., between about 10 wt % and about 40 wt %); squalane (e.g., between about 0.1 wt % and about 5 wt %); sodium hyaluronate (e.g., between about 0.1 wt % about 5 wt %); tocopherol (e.g., between about 0.01 wt % and about 2 wt %); linalool; limonene; geraniol; and citric acid; mixed with suitable active cosmetic ingredients and non-medicinal cosmetic ingredients and aqua (water), as appropriate for the intended purpose.

In some embodiments, the composition is in the form of a cream or serum suitable for use in skin care, e.g., on the face. In an embodiment, the composition also includes parfum (fragrance), such as, without limitation, the fragrance in Morning Expert™ cosmetics and personal care products (commercially available from ThisWorks®, Wimbledon, UK).

Test Composition B:

In another embodiment, the composition may include between about 0.5 wt % and about 10 wt % CBD; ascorbyl glucoside (e.g., between about 5 wt % and about 35 wt %); sodium hyaluronate (e.g., between about 0.1 wt % about 5 wt %); caprylic/capric triglyceride (e.g., between about 10 wt % and about 40 wt %); squalane (e.g., between about 0.1 wt % and about 5 wt %); arginine (e.g., between about 1 wt % and about 20 wt %); linalool; limonene; geraniol; and tocopherol (e.g., between about 0.01 wt % and about 2 wt %); mixed with other suitable active cosmetic ingredients and non-medicinal cosmetic ingredients and aqua (water), as appropriate for the intended purpose. In some embodiments, the composition is in the form of a cream or a serum for use in skin care, e.g., on the face.

In an embodiment, the composition also includes parfum (fragrance), such as, without limitation, the fragrance in Morning Expert™ cosmetics and personal care products (commercially available from ThisWorks®, Wimbledon, UK).

Test Composition C:

In another embodiment, the composition may include between about 0.5 wt % and about 10 wt % CBD; bakuchiol (e.g., between about 0.5 wt % and about 15 wt %); caprylic/capric triglyceride (e.g., between about 10 wt % and about 40 wt %); tocopherol (e.g., between about 0.01 wt % and about 2 wt %); squalane (e.g., between about 0.1 wt % and about 5 wt %); one or more essential oil (e.g., Cinnamomum camphora (Camphor) Leaf Oil, Lavandula angustifolia (Lavender) Oil, Ormenis multicaulis (Chamomile) Flower Extract, Pogostemon cablin (Patchouli) Oil, Vetiveria zizanoides (Vetivert) Root Oil, or a combination thereof); linalool; limonene; and geraniol; mixed with other suitable active cosmetic ingredients and non-medicinal cosmetic ingredients and aqua (water), as appropriate for the intended purpose. In some embodiments, the composition is in the form of a cream or a serum for use in skin care, e.g., on the face.

Test Composition D

In another embodiment, the composition may include between about 0.5 wt % and about 10 wt % CBD; hydroxypinacolone retinoate (e.g., between about 0.01 wt % and about 0.5 wt %); caprylic/capric triglyceride (e.g., between about 10 wt % and about 40 wt %); tocopherol (e.g., between about 0.01 wt % and about 2 wt %); squalane (e.g., between about 0.1 wt % and about 5 wt %); one or more essential oil (e.g., Cinnamomum camphora (Camphor) Leaf Oil, Lavandula angustifolia (Lavender) Oil, Ormenis multicaulis (Chamomile) Flower Extract, Pogostemon cablin (Patchouli) Oil, Vetiveria zizanoides (Vetivert) Root Oil, or a combination thereof); linalool; limonene; and geraniol; mixed with other suitable active cosmetic ingredients and non-medicinal cosmetic ingredients and aqua (water), as appropriate for the intended purpose. In some embodiments, the composition is in the form of a cream or a serum for use in skin care, e.g., on the face.

Test Composition E:

In another embodiment, the composition may include between about 0.5 wt % and about 10 wt % CBD; one or more alpha hydroxy acid (AHA) (e.g., between about 1 wt % and about 10 wt %); caprylic/capric triglyceride (e.g., between about 10 wt % and about 40 wt %); tocopherol (e.g., between about 0.01 wt % and about 2 wt %); squalane (e.g., between about 0.1 wt % and about 5 wt %); one or more essential oil (e.g., Boswellia carterii (Frankincense) Gum Oil, Cinnamomum camphora (Camphor) Leaf Oil, Citrus aurantium amara (Bitter Orange) Leaf Oil, Cymbopogon martini (Palmarosa) Oil, Eucalyptus globulus Leaf Oil, Pogostemon cablin (Patchouli) Oil, or a combination thereof); linalool; limonene; citral, farnesol, and geraniol; one or more amino acid (e.g., arginine) (e.g., between about 0.1 wt % and about 5 wt %); mixed with other suitable active cosmetic ingredients and non-medicinal cosmetic ingredients and aqua (water), as appropriate for the intended purpose. In some embodiments, the composition is in the form of a cream or a serum for use in skin care, e.g., on the face.

### Example 2—Clinical Study of a Composition with CBD and Vitamin C

21 subjects were clinically assessed to see the improvement over 28 days of use of Test Composition B. Each of the subjects was assessed using the following laboratory instrumentations: Each of the subjects was assessed using the following laboratory instrumentations: Wrinkles and fine lines were measured by taking high definition photographs and using commercial software to quantify the quantity and length. Skin pigmentation, tone and erythema were assessed using SkinColorCatch, which directly measures the melanin, tone (ITA°) and erythema (redness) (Delfin Technologies, Surrey, UK). Evenness was calculated from the standard deviation taken at a minimum of 3 locations on the face, measuring skin tone using Visioface HD imaging technology (VISIA® Skin Analysis System, Canfield Scientific, Utrecht, Netherlands). Skin smoothness was assessed by Image analysis correlated with skin moisturization. Skin moisturisation/hydration was assessed by measuring the capacitance of the layered structure composed of the probe, stratum corneum and the underlying skin layers, which is directly proportional to the water content of stratum corneum. Redness (erythema) was assessed using SkinColorCatch, which directly measures erythema (redness) (Delfin Technologies, Surrey, UK).

Results: In the clinical study of 21 subjects, 38% saw a reduction in the depth of wrinkles; 81% saw a reduction in the area of wrinkles; 43% saw a reduction in pigmentation (melanin); 52% saw a reduction in skin redness (erythema); and 52% saw an increase in skin hydration. The results show that the composition improved pigmentation and skin tone and reduced redness after 28 days of use by the subjects and can be used to smooth, brighten and increase skin radiance.

### Example 3—Clinical Study of a Composition with CBD and Co-Enzyme Q10

20 subjects were clinically assessed to see the improvement over 28 days of use of Test Composition A. Each of the subjects was assessed using the following laboratory instrumentations: Wrinkles and fine lines were measured by taking high definition photographs and using commercial software to quantify the quantity and length. Skin pigmentation, tone and erythema were assessed using SkinColorCatch, which directly measures the melanin, tone (ITA°) and erythema (redness) (Delfin Technologies, Surrey, UK). Evenness was calculated from the standard deviation taken at a minimum of 3 locations on the face, measuring skin tone using Visioface HD imaging technology (VISIA® Skin Analysis System, Canfield Scientific, Utrecht, Netherlands). Skin smoothness was assessed by Image analysis correlated with skin moisturisation (Schrader & Bielefeldt, 1991). Skin moisturisation/hydration was assessed by measuring the capacitance of the layered structure composed of the probe, stratum corneum and the underlying skin layers, which is directly proportional to the water content of stratum corneum. Redness (erythema) was assessed using SkinColorCatch, which directly measures erythema (redness) (Delfin Technologies, Surrey, UK).

Results: In the clinical study of 20 subjects, 52% saw a reduction in the depth of wrinkles; 62% saw a reduction in the area of wrinkles; 57% saw a reduction in pigmentation (melanin); 52% saw a reduction in skin redness (erythema); and 52% saw an increase in skin hydration. The results show that the composition improved pigmentation and skin tone and reduced redness after 28 days of use by the subjects and can be used to smooth, brighten and increase skin radiance.

### Example 4—Clinical Study of a Composition with CBD and Granactive Retinoid

21 subjects were clinically assessed to see the improvement over 28 days of use of Test Composition D. Each of the subjects was assessed using the following laboratory instrumentations: Skin elasticity was assessed by imposing a constant deformation against a reference plate when in full contact with the skin. Skin smoothness was assessed by Image analysis correlated with skin moisturisation (Schrader & Bielefeldt, 1991). Wrinkles and fine lines were measured by taking high definition photographs and using commercial software to quantify the quantity and length. Skin moisturisation/hydration was assessed by measuring the capacitance of the layered structure composed of the probe, stratum corneum and the underlying skin layers, which is directly proportional to the water content of stratum corneum. Inflammation (erythema) was assessed using SkinColorCatch, which directly measures erythema (redness) (Delfin Technologies, Surrey, UK).

Results: In the clinical study of 21 subjects, 62% saw an improvement in skin elasticity; 33% saw a reduction in the depth of wrinkles; 57% saw a reduction in the area of wrinkles; 71% saw a reduction in skin redness (erythema); and 43% saw an increase in skin hydration. The results show that the composition improved skin elasticity and fairness and reduced the appearance of fine lines and wrinkles after 28 days of use by the subjects and can be used to smooth, brighten and improve the appearance of the skin.

### Example 5—Clinical Study of a Composition with CBD and AHA Complex

20 subjects were clinically assessed to see the improvement over 28 days of use of Test Composition E. Each subject was assessed using the following laboratory instrumentations: (a) Skin clearance was assessed by measuring the skin sebum using the Sebum meter, which directly measures the amount of sebum expressed. (b) Skin brightness, tony and erythema were assessed using SkinColorCatch, which directly measures the brightness (L*a*b*), tony (ITA°) and erythema (redness) (Delfin Technologies, Surrey, UK). Evenness of tone was calculated from the standard deviation taken at a minimum of 3 locations on the face. Blemishes and skin tone were assessed using Visioface HD imaging technology (VISIA® Skin Analysis System, Canfield Scientific, Utrecht, Netherlands). Skin moisturization/hydration was assessed by measuring the capacitance of the layered structure composed on the probe, stratum corneum and the underlying skin layers, which is directly proportional to the water content of stratum corneum.

The composition can be used to exfoliate the skin to improve clarity, clear the complexion, and even the skin tone.

### Example 6—Clinical Study of a Composition with CBD and Bakuchiol

20 subjects were clinically assessed to see the improvement over 28 days of use of Test Composition C. Each subject was assessed using the following laboratory instrumentations: Skin elasticity was assessed by imposing a constant deformation against a reference plate when in full contact with the skin. Skin smoothness was assessed by Image analysis correlated with skin moisturisation (Schrader & Bielefeldt, 1991). Wrinkles and fine lines were measured by taking high definition photographs and using commercial software to quantify the quantity and length. Skin moisturization/hydration were determined by measuring the capacitance of the layered structure composed of the probe, stratum corneum and the underlying skin layers, which is directly proportional to the water content of stratum corneum. Inflammation (erythema) was assessed using SkinColorCatch, which directly measures erythema (redness) (Delfin Technologies, Surrey, UK).

The results show that the composition improved pigmentation and skin tone and reduced redness after 28 days of use by the subjects and can be used to smooth, brighten and increase skin radiance.

The composition can be used to smooth, brighten and improve the appearance of the skin.

### INCORPORATION BY REFERENCE

All references cited in this specification, and their references, are incorporated by reference herein in their entirety where appropriate for teachings of additional or alternative details, features, and/or technical background.

### EQUIVALENTS

While the disclosure has been particularly shown and described with reference to particular embodiments, it will be appreciated that variations of the above-disclosed and other features and functions, or alternatives thereof, may be desirably combined into many other different systems or applications. Also, that various presently unforeseen or unanticipated alternatives, modifications, variations or improvements therein may be subsequently made by those skilled in the art which are also intended to be encompassed by the following embodiments.

1. A composition comprising:
at least one cannabinoid or a derivative thereof; and
at least one active cosmetic ingredient.

2. The composition according to claim 1, further comprising at least one non-medicinal cosmetic ingredient.

3. The composition according to claim 2, wherein the at least one non-medicinal cosmetic ingredient is an antioxidant.

4. The composition according to claim 3, wherein the antioxidant is tocopherol.

5. The composition according to claim 1, further comprising a functional fragrance.

6. (canceled)

7. The composition according to claim 1, wherein the at least one cannabinoid is: cannabigerol ((E)-CBG C-5), cannabigerol monomethyl ether ((E)-CBGM C-5A), Cannabigerolsäure A ((Z)-CBGA C-5A), Cannabigerovarin (((e)-CBGV C-3), Cannabigerolsäure A (e)-CBGA C-5A), A Cannabigerolsäure monomethyl ether ((e)-CBGAM C-5A), Cannabigerovarinsäure A ((e)-CBGVA-C3A); cannabichromene (CBC-C5), Cannabichromensäure A (CBCA C-5A), Cannabichromevarin (CBCVC-3), Cannabichromevarinsäure A (CBCVA-C3A); cannabidiol (CBD-C5), cannabidiol monomethyl (CBDM-C5), cannabidiol-C4 (CBD-C4), Cannabidivarin (CBDV-C3), Cannabidiorcol (CBD-C1), cannabidiolic (CBDA C-5), Cannabidivarinsäure (CBDVA C-3); Cannabinodiol-like (CBND): Cannabinodiol (CBND C-5), Cannabinodivarin (CBND C-3); Tetrahydrocannabinol-like (THC): Δ⁹-tetrahydrocannabinol (Δ⁹-THC-C5), Δ⁹-tetrahydrocannabinol-C4 (Δ⁹-THC-C4), Δ⁹-tetrahydrocannabivarin (Δ⁹-THCV-C3), Δ9-Tetrahydrocannabiorcol (Δ⁹-THCO-C-1), Δ⁹-Tetrahydrocannabinolsäure (Δ⁹THCA-C-5A), Δ⁹-Tetrahydrocannabinolsäure B (Δ⁹THCA-C-5B), Δ⁹-Tetrahydrocannabinolsäure-C4 (Δ⁹THCA-C-4A and/or B), Δ⁹-Tetrahydrocannabivarinsäure A (Δ⁹-THCVA-C3A), Δ⁹-Tetrahydrocannabiorcolsäure (Δ⁹-THCOA-C1 A and/or B), (+Δ⁸-trans-(6aR,10aR)-Δ⁸-tetrahydrocannabinol(Δ⁸-THC-C5), (+Δ⁸-trans-(6aR,10aR)-Tetrahydrocannabinolsäure A (Δ⁸-THCA-C5 A); (−)-(6aS,10aR)-Δ⁹-tetrahydrocannabinol ((−)-cis-Δ⁹-THC-C5); Cannabinol CBN-C5, cannabinol C4 (CBN-C4), Cannabivarin (CBN-C3), cannabinol C2 (CBN-C2), Cannabiorcol (CBN-C1), Cannabinolsäure A (C5 CBNA-A), Cannabinolmethylether (CBNM C-5) Cannabitriol-type (CBT): (−)-(9R,10R)-trans-Cannabitriol ((−)-trans-CBT-C5), (+)-(9S,10S)-Cannabitriol ((+)-trans-CBT C-5), (±)-(9R,10S/9S, 10R)-Cannabitriol ((±)-cis-CBT-C5), (−)-(9R,10R)-trans [10-0-ethyl-cannabitriol] ((−)-trans-CBT-OEt-C5), (±)-(9R,10R/9S,10S)-Cannabitriol-C3 ((±)-trans-CBT-C3), 8,9-dihydroxy-Δ6a (10a) tetrahydrocannabinol (8,9-di-OH-CBT-C5), cannabidiolic A (CBDA C-59-OH-CBT-C5 ester), (−)-(6aR,9S,10S,10aR)-9,10-dihydroxy-hexahydrocannabinol, Cannabiripsol Cannabiripsol-C5, (−)-6a,7,10a-trihydroxy-Δ⁹-tetrahydrocannabinol ((−)-Cannabitetrol), 10-oxo-Δ6a (10a) tetrahydrocannabinol (OTHC); Cannabielsoin-like (CBE): (5aS, 6S, 9R, 9aR)-C5-Cannabielsoin (CBEC-5), (5aS, 6S, 9R, 9aR)-C3-Cannabielsoin (CBE C-3), (5aS, 6S, 9R, 9aR)-Cannabielsoinsäure A (CBEA-C5A), (5aS,6S,9R,9aR)-Cannabielsoinsäure B (CBEA-C5B), (5aS, 6S, 9R, 9aR)-C3-Cannabielsoinsäure B (CBEA-C3B), Cannabiglendol-C3 (OH-iso-HHCV C-3), Dehydrocannabifuran (DCBF C-5), Cannabifuran (CBF-C5); Isocannabinoide: (+Δ⁷-trans-(1R,3R,6R)-Isotetrahydrocannabinol, (±)-Δ7-1,2-cis-(1R,3R,6S/1S,3S,6R)-Isotetrahydro-cannabivarin, (+Δ⁷-trans-(1R,3R,6R)-Isotetrahydrocannabivarin; (±)-(1aS,3aR,8bR,8Cr-cannabicyclol (CBL-C5), (±)-(1aS,3aR, 8bR,8Cr-Cannabicyclolsäure A (CBLA-C5A) (±)-(1aS,3aR,8bR,8Cr-Cannabicyclovarin (CBLV C-3); Cannabicitran-type (CBT): Cannabicitran (CBT-C5); Cannabichromanon (CBCN C-5), Cannabichromanon-C3 (CBCN C-3), or Cannabicoumaronon (CBCON C-5).

8. The composition according to claim 1, wherein the at least one cannabinoid is cannabidiol (CBD).

9.-10. (canceled)

11. The composition according to claim 1, wherein the at least one cannabinoid is present in the composition in an amount ranging from about 0.5 wt % to about 10 wt %.

12. (canceled)

13. The composition according to claim 3, wherein the antioxidant is present is present in the composition in an amount ranging from about 0.05 wt % to about 1 wt %.

14. (canceled)

15. The composition according to claim 1, wherein the at least one active cosmetic ingredient is selected from: Ascorbyl Glucoside, Ubiquinone, one or more alpha hydroxy acid (AHA), Bakuchiol, Hydroxypinacolone Retinoate (HPR), and combinations thereof.

16. The composition according to claim 15, wherein the one or more AHA is selected from glycolic acid, citric acid, tartaric acid, lactic acid, and combinations thereof.

17. The composition according to claim 15, wherein the composition further comprises one or more of: Caprylic/Capric Triglyceride, Squalane, Sodium hyaluronate, one or more amino acid, sodium lactate, glycerin, Aloe vera, Aloe barbadensis Leaf Juice Powder, one or more essential oil, Glyceryl stearate, Octyldodecanol, PCA, Sodium PCA, ethylhexylglycerin, lecithin, BHA, BHT, derivatives thereof, and combinations thereof.

18. The composition according to claim 1, wherein the composition further comprises at least one purified terpene.

19. The composition according to claim 18, wherein the at least one purified terpene is selected from: linalool, linalool oxide, limonene, geraniol, citronellol, eugenol, citral, farnesol, derivatives thereof, and combinations thereof.

20. The composition according to claim 17, wherein the one or more essential oil is selected from: Vitis vinifera Seed (Grapeseed) Oil; Lavandula angustifolia (Lavender) Oil; Cinnamomum camphora Leaf (Ho Wood) Oil; Pogostemon cablin (Patchouli) Oil; Vetiveria zizanoides Root (Vetiver) Oil; Chamaemelum nobile Flower (Roman Chamomile) Oil; Cocos nucifera (Coconut) Oil; Prunus amygdalus dulcis (Sweet Almond) Oil; Simmondsia chinensis (Jojoba) Seed Oil; and combinations thereof.

21. The composition according to claim 17, wherein the one or more amino acid is selected from: Arginine, Aspartic Acid, Glycine, Alanine, Serine, Valine, Isoleucine, Proline, Threonine, Histidine, Phenylalanine, and combinations thereof.

22. The composition according to claim 1, wherein the composition comprises one or more of: Zingiber officinale (ginger) root extract; Cinnamomum camphora (camphor) leaf oil; Lavandula angustifolia (lavender) oil; Ormenis multicaulis (chamomile) flower extract; Pogostemon cablin (patchouli) oil; Vetiveria zizanoides (vetivert) root oil; Boswellia carterii (frankincense) gum oil; Cinnamomum camphora (camphor) oil; citrus Aurantium amara (bitter orange) leaf oil; Cymbopogon martini (palmarosa) oil; Eucalyptus globulus leaf oil; and combinations thereof.

23. The composition according to claim 1, wherein the composition further comprises one or more of: Phenoxyethanol, Sodium Metabisulfite, Dimethyl Isosorbide, Tartaric Acid, Magnesium Chloride, Sodium stearoyl glutamate, Benzyl alcohol, Butylene glycol, Xanthan Gum, Polysorbate 20, Disodium EDTA, Sodium hydroxide, Dehydroacetic Acid, Stearic acid, Cetyl alcohol, Ceteth-20, Steareth-20, PEG-75 Stearate, C10-30 Alkyl Acrylate Crosspolymer, Parfum (fragrance), derivatives thereof, and combinations thereof.

24. The composition according to claim 1, wherein the composition is in the form of an ointment, a cream, a suspension, a liniment, a balm, a serum, a lotion, a paste, a gel, a spray, an aerosol, a foam, an oil, a liquid, a powder, a patch, a matrix, or a wax.

25.-27. (canceled)

28. A cosmetic or a personal care product comprising the composition according to claim 1.

29.-37. (canceled)
